(19) United States
(12) Patent Application Publication
(10) Pub. No.: US 2023/0277312 A1
(43) Pub. Date: Sep. 7, 2023

(54) PROSTHETIC HEART VALVES WITH SEALING FRAMES TO REDUCE PARAVALVULAR LEAKAGE

(71) Applicant: EDWARDS LIFESCIENCES CORPORATION, Irvine, CA (US)

(72) Inventors: Tram Ngoc Nguyen, Santa Ana, CA (US); Hannah Reed Bettencourt, Huntington Beach, CA (US); Evan T. Schwartz, Huntington Beach, CA (US)

(21) Appl. No.: 18/315,500

(22) Filed: May 11, 2023

Related U.S. Application Data
(63) Continuation of application No. PCT/US21/58790, filed on Nov. 10, 2021.
(60) Provisional application No. 63/112,567, filed on Nov. 11, 2020.

(51) Int. Cl. A61F 2/24

(52) U.S. Cl. CPC A61F 2/2418; A61F 2/2466; A61F 2250/0069; A61F 2210/0014

(57) ABSTRACT

A sealing frame surrounds a radially-outer surface portion of a valve frame of a prosthetic heart valve. Both the valve frame and the sealing frame are radially collapsible and expandable between respective compressed and expanded configurations. The sealing frame has a first axial end coupled to the valve frame at its inflow end, and a second axial end coupled to the valve frame at a location between its inflow and outflow ends. The sealing frame also has an intermediate portion between the first and second axial ends that projects radially outward when the valve and sealing frames are in their expanded configurations. The sealing frame displaces an outer skirt of the prosthetic heart valve radially outward and urges the outer skirt into contact with surrounding native tissue, thereby reducing or avoiding paravalvular leakage. In some examples, the sealing frame can be formed of a shape memory material.

### CROSS-REFERENCE TO RELATED APPLICATIONS

This application is a continuation of PCT patent application no. PCT/US2021/058790, filed on Nov. 10, 2021, which claims the benefit of U.S. Provisional Application No. 63/112,567 filed Nov. 11, 2020, each of which is incorporated herein in its entirety by this specific reference.

### FIELD

The present disclosure relates to prosthetic heart valves, in particular, prosthetic heart valves with sealing frames to reduce paravalvular leakage.

### BACKGROUND

The human heart can suffer from various valvular diseases, which can result in significant malfunctioning of the heart and ultimately require repair of the native valve or replacement of the native valve with an artificial valve. There are a number of known repair devices (e.g., annuloplasty rings) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped configuration on an end of a delivery device and advanced through the patient's vasculature until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size.

Such expandable, transcatheter heart valves can have an annular frame, a valvular structure formed by a plurality of leaflets supported within the frame, and one or more skirt coupled to an interior of the frame, and an outer skirt coupled to an exterior of the frame. The outer skirt help reduce paravalvular leakage (PVL) by obstructing any flowpath that may exist between the exterior of the frame and the surrounding native tissue. In general, the outer skirt is formed of a fabric or material covering at least a portion of the frame exterior. However, because such outer skirts lack any type of internal support, some anatomical features may limit the effectiveness in preventing PVL. For example, when parts of the patient's anatomy in which the valve is implanted are relatively stiff (e.g., due to calcification nodules) and/or when a shape of the anatomy extends beyond a size of the implanted valve frame (e.g., due to the relatively large commissural gaps of the native mitral valve), the outer skirt may be unable to close off gaps between the valve frame exterior and the surrounding native tissue.

Accordingly, a need exists for prosthetic heart valves that reduce the risk of thrombosis from implantation thereof, and methods for implanting and assembling such prosthetic heart valves.

### SUMMARY

In order to reduce paravalvular leakage (PVL), a prosthetic heart valve can be provided with an expandable sealing frame. The sealing frame can surround an exterior surface portion of an expandable frame of the prosthetic heart valve. When the sealing frame and valve frame are in their expanded configurations, a portion of the sealing frame can protrude radially outward from the valve frame, thereby urging an outer skirt of the prosthetic heart frame radially outward. Whereas conventional outer skirts lack underlying support and therefore may be unable to overcome relatively stiff anatomy (e.g., due to calcification) or to extend over relatively large distances (e.g., due to large commissural gaps or irregularly-shaped annuli), the sealing frames disclosed herein can push the outer skirt into contact with surrounding native tissue, thereby closing any gaps between the prosthetic heart valve and the patient's anatomy that may have otherwise contributed to PVL.

In a representative example, axial ends of the sealing frame are attached to the valve frame, and foreshortening of the valve frame (e.g., a reduction in height along its axial direction) as it expands to the expanded configuration can urge an intermediate portion of the sealing frame between the axial ends to deflect radially outward (e.g., by buckling or bending). In some examples, the expandable sealing frame is constructed of a shape memory material (e.g., nickel titanium alloy, such as Nitinol), and a pre-deformed shape of the expanded sealing frame has an intermediate portion that deflects radially outward. In such configurations, the sealing frame may be deformed into a compressed configuration for delivery to an implantation site, and the foreshortening of the valve frame at the implantation site may assist the sealing frame in reverting to the desired pre-deformed shape with the intermediate portion projecting radially outward.

In another representative example, a prosthetic heart valve can comprise a valve frame, a valvular structure, a sealing frame, and an outer skirt. The valve frame can be radially collapsible and expandable between a first compressed configuration and a first expanded configuration. The valve frame can have an inflow end and an outflow end separated from the inflow end along an axial direction of the valve frame. The valvular structure can be coupled to the valve frame and comprise a plurality of leaflets within the valve frame. The sealing frame can surround a radially-outer surface portion of the valve frame. The sealing frame can be collapsible and expandable between a second compressed configuration corresponding to the first compressed configuration of the valve frame and a second expanded configuration corresponding to the second expanded configuration of the valve frame. The sealing frame can have a first axial end coupled to the valve frame at the inflow end, a second axial end coupled to the valve frame at a location between the inflow and outflow ends along the axial direction, and an intermediate portion between the first and second axial ends along the axial direction. The outer skirt can surround the sealing frame. With the valve frame and the sealing frame in the first and second expanded configurations, respectively, the intermediate portion can project radially outward from the valve frame, thereby displacing at least a portion of the outer skirt radially outward.

In another representative example, a prosthetic heart valve can comprise a valve frame, a valvular structure, an outer skirt, and means for displacing at least a portion of the outer skirt radially outward from the valve frame. The valve frame can be radially collapsible and expandable between a compressed configuration and an expanded configuration. The valve frame can have an inflow end and an outflow end separated from the inflow end along an axial direction of the valve frame. The valvular structure can be coupled to the valve frame and can comprise a plurality of leaflets within the valve frame.

In another representative example, a method of assembling a prosthetic heart valve can comprise coupling a sealing frame to a radially-outer surface of a valve frame of the prosthetic heart valve. The valve frame can be radially collapsible and expandable between a first compressed configuration and a first expanded configuration. The valve frame can have an inflow end and an outflow end separated from the inflow end along an axial direction of the valve frame. The sealing frame can be collapsible and expandable between a second compressed configuration corresponding to the first compressed configuration of the valve frame and a second expanded configuration corresponding to the second expanded configuration of the valve frame. The sealing frame can have a first axial end, a second axial end, and an intermediate portion between the first and second axial ends along the axial direction. The coupling can be such that the first axial end is coupled to the valve frame at the inflow end and the second axial end coupled is to the valve frame at a location between the inflow and outflow ends along the axial direction. The method can further comprise providing an outer skirt surrounding the radially-outer surface of the valve frame. With the valve frame and the sealing frame in the first and second expanded configurations, respectively, the intermediate portion can project radially outward from the valve frame, thereby displacing at least a portion of the outer skirt radially outward.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE DRAWINGS

FIG. 1 shows a schematic cross-sectional view of a human heart in which a prosthetic heart valve may be installed.

FIG. 2 shows a schematic top view of a mitral valve annulus of a heart.

FIG. 3A is a perspective view from an outflow end of a configuration of an exemplary prosthetic heart valve.

FIG. 3B is a perspective view from an outflow end of an annular frame for the exemplary prosthetic heart valve of FIG. 3A.

FIG. 3C is a simplified cross-sectional side view of the prosthetic heart valve of FIG. 3A implanted at the mitral position in a patient's heart using a docking device.

FIG. 4A is a simplified cross-sectional side view of an exemplary prosthetic heart valve with an exemplary sealing frame in respective expanded configurations.

FIG. 4B is a simplified cross-sectional side view of the prosthetic heart valve and sealing frame of FIG. 4A in respective compressed configurations.

FIG. 4C is a perspective view from an outflow end of a configuration of the prosthetic heart valve of FIG. 3A employing a sealing frame to expand an outer skirt.

FIG. 4D is a simplified cross-sectional side view of the prosthetic heart valve of FIG. 4C implanted at the mitral position in a patient's heart using a docking device.

FIG. 5A is a side perspective view of a first exemplary sealing frame in an expanded configuration.

FIG. 5B is a simplified cross-sectional view illustrating interaction between an outer skirt and the sealing frame of FIG. 5A coupled to a prosthetic valve frame.

FIG. 5C is a simplified flat view of an assembly comprising an outer skirt coupled to the sealing frame of FIG. 5A.

FIG. 5D is a simplified flat view of an exterior of a prosthetic heart valve employing the assembly of FIG. 5C.

FIG. 5E is a simplified flat view of an exterior of a prosthetic heart valve employing the sealing frame of FIG. 5A and an outer skirt separately coupled to the valve frame.

FIGS. 5F-5G are simplified flat views of an exterior of a prosthetic heart valve employing a first variation and a second variation, respectively, for the assembly of FIG. 5C.

FIG. 6A is a side perspective view of a second exemplary sealing frame in an expanded configuration.

FIG. 6B is a simplified flat view of an assembly comprising an outer skirt coupled to the sealing frame of FIG. 6A.

FIG. 7A is a side perspective view of a third exemplary sealing frame in an expanded configuration.

FIG. 7B is a simplified flat view of an assembly comprising an outer skirt coupled to the sealing frame of FIG. 7A.

FIG. 7C is a simplified flat view of an exterior of a prosthetic heart valve employing the assembly of FIG. 7B.

FIG. 7D is a simplified flat view of an exterior of a prosthetic heart valve employing a variation of the sealing frame of FIG. 7A and an outer skirt separately coupled to the valve frame.

FIG. 8A is a side perspective view of a fourth exemplary sealing frame in an expanded configuration.

FIG. 8B is a simplified flat view of an assembly comprising an outer skirt coupled to the sealing frame of FIG. 8A.

FIG. 8C is a simplified flat view of an exterior of a prosthetic heart valve employing the assembly of FIG. 8B.

FIG. 9 is a simplified view of an exemplary delivery system for implanting a prosthetic heart valve within a patient.

### DETAILED DESCRIPTION

Described herein are prosthetic heart valves that employ sealing frames to reduce paravalvular leakage (PVL). In some examples, the sealing frame can surround an exterior surface portion of the valve frame adjacent to an inflow end of the prosthetic valve, and the sealing frame can be disposed between an outer skirt and the valve frame. Both the valve frame and the sealing frame can be radially collapsible and expandable between respective compressed (e.g., crimped state) and expanded (e.g., deployed state) configurations. When in its expanded configuration, a portion of the sealing frame can protrude radially outward from the valve frame. The protruding portion of the sealing frame can also expand the outer skirt radially outward or at least apply a radially-outwardly-directed force to the outer skirt, thereby urging the outer skirt into contact with surrounding native tissue. Thus, any gaps between the prosthetic heart valve and the patient's anatomy, which gaps may have otherwise contributed to PVL, can be closed by the expanded outer skirt.

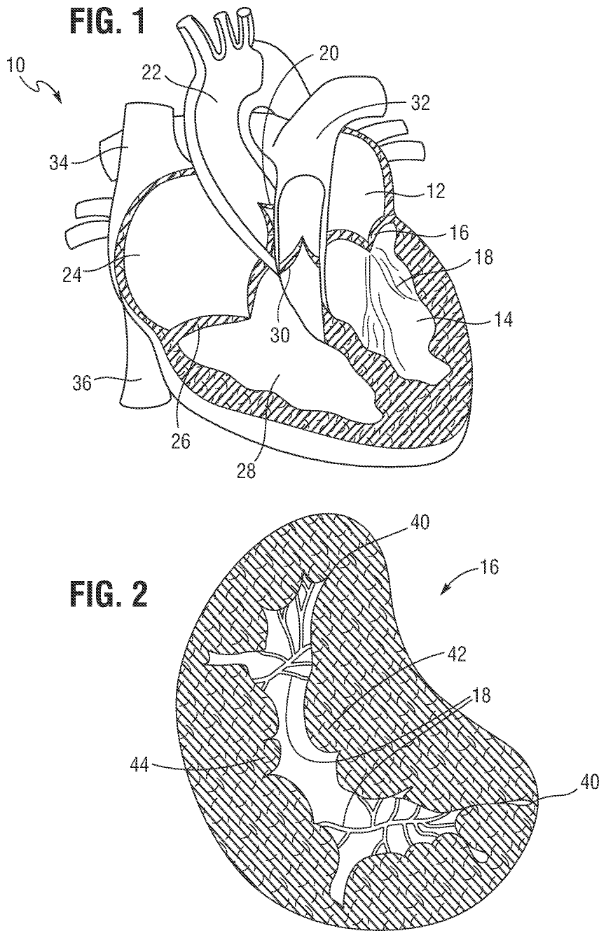

Referring to FIG. 1, a schematic cross-sectional view of a human heart 10 is shown. The mitral valve 16 separates the left ventricle 14 from the left atrium 12, and the tricuspid valve 26 separates the right ventricle 28 from the right atrium 24. The aortic valve 20 further separates the left ventricle 14 from the ascending aorta 22, and the pulmonary valve 30 further separates the right ventricle 28 from the pulmonary artery 32. Deoxygenated blood is delivered to the right atrium 24 by the superior vena cava 34, the inferior vena cava 36, and the coronary sinus. During the diastolic phase, as the right ventricle 28 expands, deoxygenated blood in the right atrium 24 is directed through the tricuspid valve 26 into the right ventricle 28. In the subsequent systolic phase, contraction by the right ventricle 28 forces the deoxygenated blood therein through the pulmonary valve 30 into the pulmonary artery 32. In addition to forcing blood through the one-way pulmonary valve 30, the pressure of the contraction by the right ventricle 28 also urges the one-way tricuspid valve 26 closed, thereby preventing blood in the right ventricle 28 from re-entering the right atrium 24.

Oxygenated blood is delivered to the left atrium 12 by the pulmonary veins. During the diastolic phase, as the left ventricle 14 expands, the oxygenated blood in the left atrium 12 is directed through the mitral valve 16 into the left ventricle 14. In the subsequent systolic phase, contraction by the left ventricle 14 forces the oxygenated blood through the aortic valve 20 into the ascending aorta 22 for circulation through the body. In addition forcing the blood through the one-way aortic valve 20, the pressure of the contraction by the left ventricle 14 also urges the one-way mitral valve 16 closed, thereby preventing blood in the left ventricle 14 from re-entering the left atrium 12. The contraction by the left ventricle 14 generates a significant pressure differential between the left ventricle 14 and the left atrium 12. A series of chordae tendineae 18 connect leaflets of the mitral valve 16 to papillary muscles located on the walls of the left ventricle 14. During the diastolic phase, both the chordae tendineae 18 and the papillary muscles are tensioned to hold the leaflets of the mitral valve 16 in the closed position and to prevent the leaflets from extending backward into the left atrium 12.

Any of the above noted native heart valves may fail to operate properly, for example, by allowing blood to backflow therethrough or regurgitate into an upstream heart chamber or blood vessel. In some examples, a prosthetic heart valve can be implanted within the native heart valve to help prevent or inhibit such regurgitation and/or to address any other insufficiency of the native heart valve. Any of the prosthetic heart valves disclosed herein can be implanted at or within any of these native heart valves, including the aortic valve 20, the pulmonary valve 30, the mitral valve 16, and the tricuspid valve 26, for example, to minimize or at least reduce PVL caused by relatively-stiff anatomical features of the native valve (e.g., due to calcification). However, the disclosed subject matter may also be particularly applicable to prosthetic valves implanted at or in the native tricuspid valve 26 or the native mitral valve 16, where a relatively large commissural gap may exist.

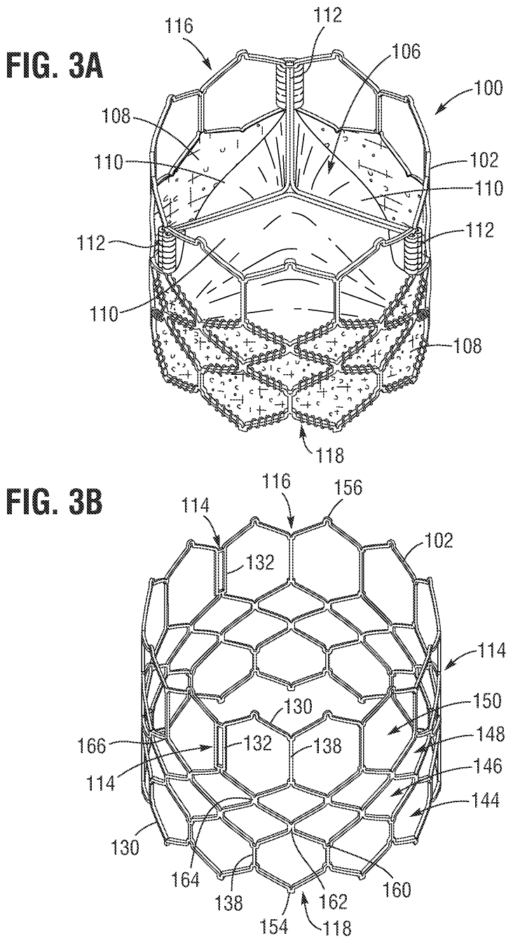

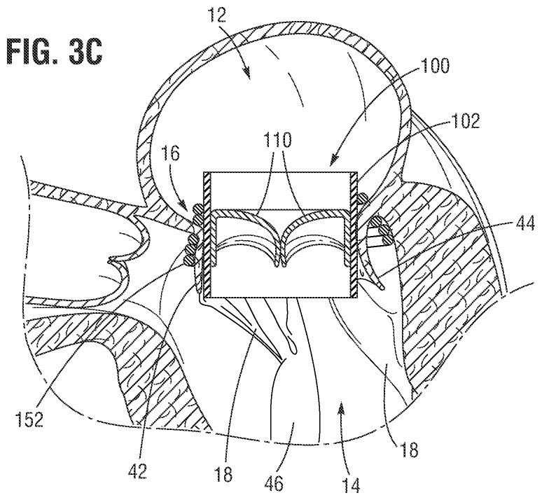

Referring to FIG. 2, a schematic top view of a mitral valve annulus is shown. The mitral valve 16 includes an anterior leaflet 42 and a posterior leaflet 44. Leaflets 42, 44 are connected to an inner wall of the left ventricle 14 via chordae tendineae 18 and papillary muscles 46 (shown in FIG. 3C). Commissures 40 are located at the ends of the mitral valve 16 where the anterior leaflet 42 and the posterior leaflet 44 come together. The annulus of the mitral valve 16 has an elongated irregular shape (e.g., reniform or bean-shaped) as compared to the more circular shape of the aortic valve 20. The shape of the mitral valve 16 can present challenges when implanting a generally-cylindrical prosthetic heart valve therein. For example, FIG. 3C shows a prosthetic heart valve 100, which has an annular frame 102 supporting a plurality of leaflets 110, implanted between native leaflets 42, 44 of mitral valve 16. If prosthetic valve 100 has a diameter that is too small to reach both commissures 40, PVL can arise through the resulting gaps; however, if prosthetic valve 100 has a diameter that is large enough to reach both commissures 40, the narrower parts of the annulus of the mitral valve 16 may otherwise be damaged. Similar challenges may present with the tricuspid valve 26, which also has an elongated irregular shape as compared to the more circular shape of the pulmonary valve 30.

In some examples, a docking station 152 (also referred to as anchoring device, docking device, or valve dock) can be installed prior to implantation of the prosthetic heart valve 100. The docking station 152 can include coils or turns that pinch or urge portions of leaflets 42, 44 inward in order to form a more circular opening for implantation of the prosthetic valve 100 therein. Further details regarding docking station configurations and methods for installation and use thereof can be found in U.S. Patent Application Publication Nos. 2018/0055628, 2018/0055630, 2018/0318079, and 2019/0192296, and International Application No. PCT/US2020/036577, all of which are incorporated herein by reference. However, even when using docking station 152, gaps may arise between the implanted valve 100 and the surrounding anatomical structures that can lead to PVL.

In some examples, a prosthetic heart valve can be provided with a sealing frame disposed between the valve frame and an outer skirt. The sealing frame can be constructed to displace the outer skirt radially outward and into contact with the surrounding anatomical structures, thereby sealing any gaps that may could contribute to PVL. Moreover, the sealing frame can provide a biasing force that urges the outer skirt into the surrounding anatomical structures, thereby overcoming any stiff tissue (e.g., due to calcification) or other stubborn anatomical features (e.g., large commissural gaps) that may have otherwise prevented the outer skirt from sealing any void spaces between the prosthetic heart valve and the surrounding anatomy.

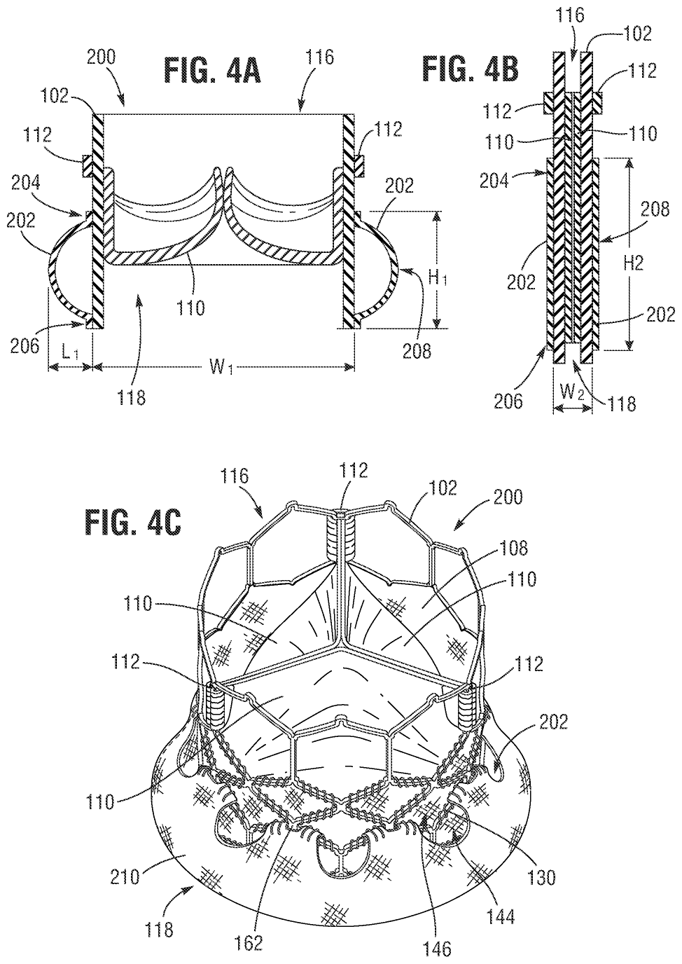

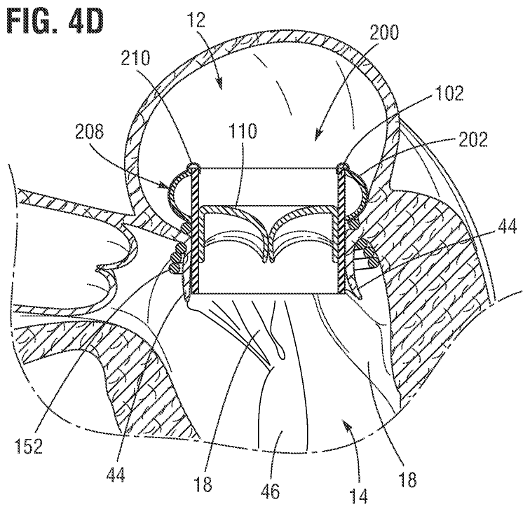

FIGS. 4A-4B illustrate a prosthetic heart valve 200 in expanded and compressed configurations, respectively. Similar to the valve illustrated in FIG. 3C, prosthetic heart valve 200 can have an annular valve frame 102 and a plurality of leaflets 110 coupled to the valve frame 102 by respective commissure assemblies 112. However, in contrast to the valve of FIG. 3C, prosthetic heart valve 200 further includes a sealing frame 202. The sealing frame 202 can surround an exterior surface portion of the valve frame 102 of the prosthetic heart valve 200. The sealing frame 202 can have a first axial end 204, a second axial end 206, and an intermediate portion 208 between the first and second axial ends. The second axial end 206 can be disposed at or substantially adjacent to an inflow end 118 of the annular frame 102. In some examples, the sealing frame 202 extends along the axial direction from the inflow end 118 of the annular frame 102 to a location between the commissures 112 and the inflow end 118, as illustrated in FIG. 4A. Alternatively, in some examples, the sealing frame 202 can extend further along a height of the valve frame 102. For example, a sealing frame may surround an entirety of the exterior surface of the annular frame 102 by extending along the axial direction from the inflow end 118 to the outflow end 116.

In some examples, the sealing frame 202 is constructed as a piece separate from the valve frame 102 and subsequently coupled thereto. For example, the first axial end 204 and the second axial end 206 can be attached to adjacent portions of the valve frame 102 (e.g., rungs or struts of the valve frame). The ends of the sealing frame 202 can be coupled to the valve frame 102 via sutures, adhesive, welding, or any other suitable attachment means. Alternatively, in some examples, the sealing frame 202 can be integrally formed with the valve frame 102. In some examples, when the sealing frame 202 and the valve frame 102 are both in their expanded configurations, the first axial end 204 and/or the second axial end 206 of the sealing frame 202 can have an inner diameter that is substantially the same as an outer diameter of the valve frame 102. Alternatively or additionally, in some examples, when the sealing frame 202 and the valve frame 102 are both in their compressed configurations, the first axial end 204 and/or the second axial end 206 of the sealing frame can have an inner diameter that is substantially the same as an outer diameter of the valve frame 102.

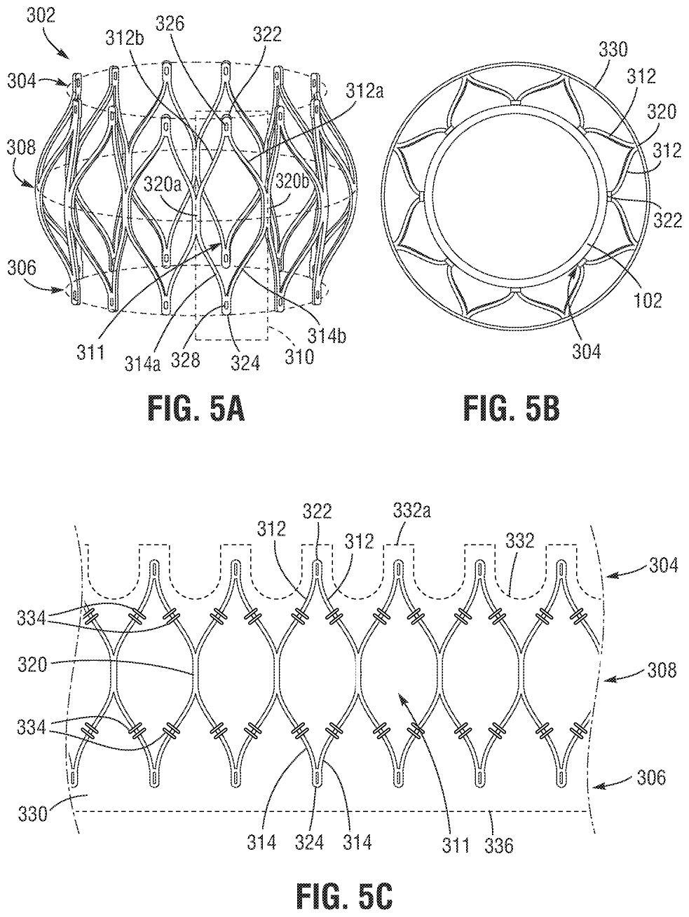

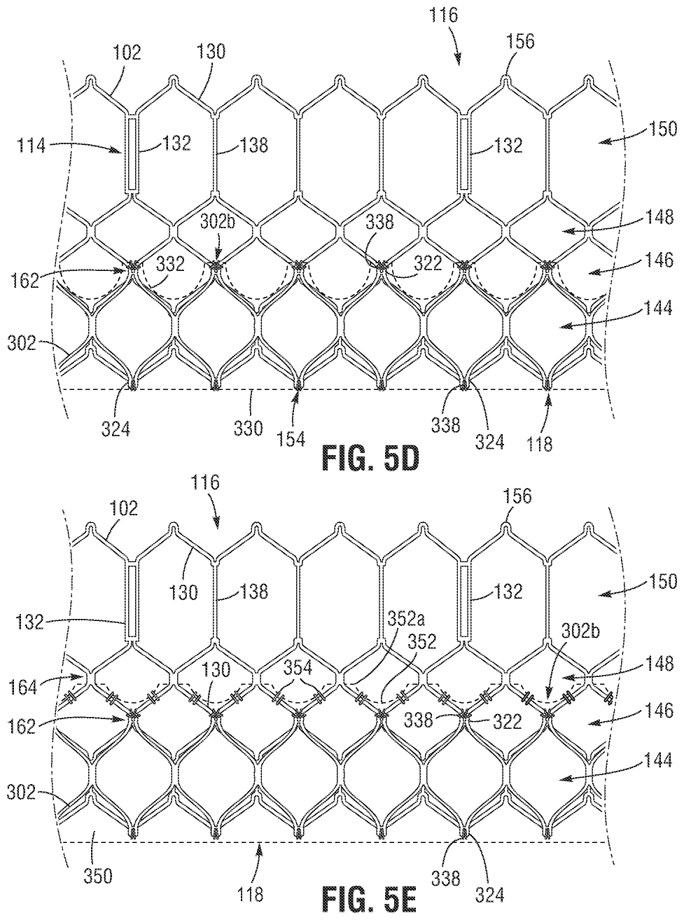

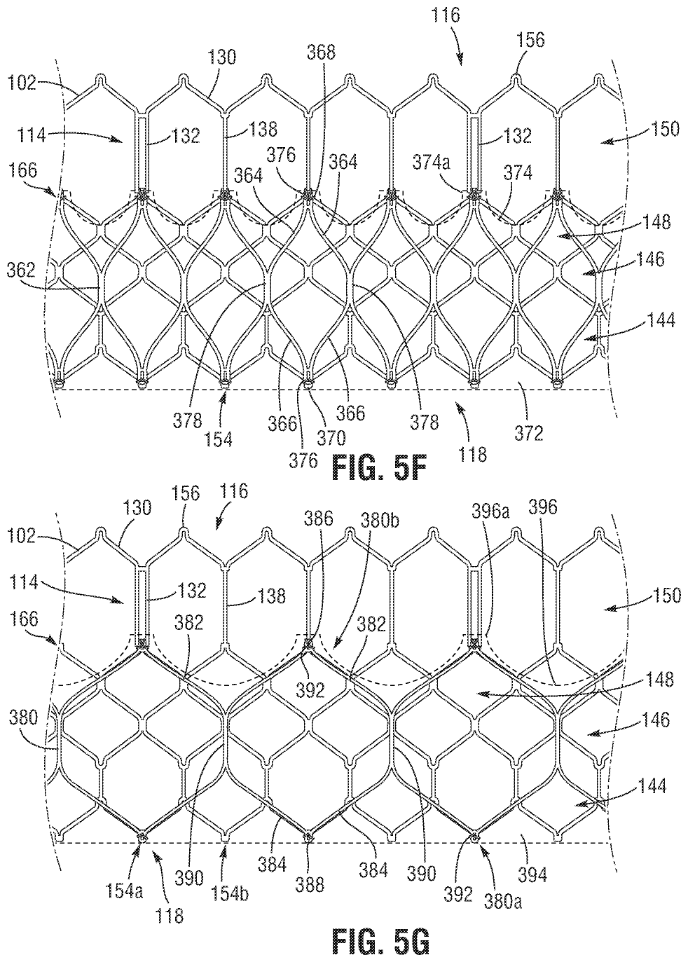

Similar to the valve frame 102, the sealing frame 202 can be constructed to be radially collapsible and expandable between a crimped or compressed configuration (shown in FIG. 4B) for delivery to the implantation site, and a deployed or expanded configuration (shown in FIG. 4A) for mounting at the implantation site. In some examples, the sealing frame 202 can be formed by a network of struts. In some examples, the struts can be connected together to form open cells (e.g., open to one of the axial ends of the sealing frame) and/or closed cells (e.g., closed to both axial ends of the sealing frame). The cells can be constructed to facilitate transition of the sealing frame 202 between the compressed configuration and the expanded configuration, for example, by collapsing in transitioning to the compressed configuration. The cells can take any of various shapes or patterns. For example, FIGS. 5A-8D, which are discussed in further detail below, illustrate various exemplary shapes and patterns for such cells; however, other shapes or patterns for cells of the sealing frame are also possible beyond those specifically illustrated.

In the compressed configuration shown in FIG. 4B, the sealing frame 202 can adopt a low-profile shape, where the first axial end 204, the second axial end 206, and the intermediate portion 208 are substantially aligned along the axial direction. For example, in the compressed configuration, the sealing frame 202 may follow a substantially-cylindrical profile and be disposed adjacent to the exterior surface of the valve frame 102. In the compressed configuration, the valve frame 102 also adopts a low-profile shape, for example, having a diameter, W₂, of about 6-8 mm. In some examples, the sealing frame 202 is constructed such that the radial size of the prosthetic heart valve in the compressed configuration is only minimally increased by the inclusion of sealing frame 202. For example, the sealing frame 202 may add no more than 1-2 mm to the overall diameter of the prosthetic heart valve 200 as compared to the diameter W₂ of the valve frame 102.

In the expanded configuration shown in FIG. 4A, the valve frame 102 increases in diameter and reduces in height. For example, the valve frame 102 can have a diameter in the expanded configuration, W₁, that is 2.5-5 times greater than the diameter in the compressed configuration, W₂, and a height in the compressed configuration that is 1.2-1.3 times greater than the height in the expanded configuration. For example, the valve frame 102 in the expanded configuration can have a diameter of 20-29 mm and a height of 15-23 mm. Since the first and second axial ends of the sealing frame 202 are coupled to the valve frame 102, the change in height of the valve frame 102 as it transitions from the compressed configuration to the expanded configuration causes the first and second axial ends of the sealing frame 202 to approach each other along the axial direction. The first axial end 204 and the second axial end 206 of the sealing frame 202 in the expanded configuration (FIG. 4A) can remain aligned along the axial direction, but a distance between the first and second axial ends is reduced as compared to the distance between the first and second axial ends of the sealing frame 202 in the compressed configuration (FIG. 4B). For example, the axial height, H₂, of the sealing frame 202 in the compressed configuration may be 1.2-1.3 times greater than the axial height, H₁, of the sealing frame 202 in the expanded configuration.

In some examples, the reduced distance between the first axial end 204 and the second axial end 206 can cause the sealing frame 202 to buckle or bend away from the valve frame 102. The sealing frame 202 in the expanded configuration thus adopts a protruding or projecting shape, where intermediate portion 208 is displaced radially outward from a corresponding exterior surface portion of the valve frame 102. In some examples, the sealing frame 202 in the expanded configuration may define or follow, in cross-section, a partial toroidal profile (e.g., formed by revolving a half-circle, partial ellipse, or any other arcuate shape about a central longitudinal axis of the valve frame 102, which axis would be coplanar with the revolved shape), as illustrated in FIG. 4A. However, other protruding profile shapes for the sealing frame 202 in the expanded configuration are also possible. For example, the sealing frame 202 in the expanded configuration may adopt an asymmetrical protruding shape, where the protruding intermediate portion 208 is closer to the first axial end 204 than the second axial end 206, or vice versa.

In some examples, the intermediate portion 208 of the sealing frame 202 extends an effective diameter of the prosthetic heart valve 200 in the expanded configuration (e.g., as defined by the diameter, W₁, of the valve frame 102) by 6-14%. For example, the intermediate portion 208 may extend along a radial direction from an exterior surface of the valve frame 102 (or from either axial end 204, 206, which may be coincident with the valve frame exterior surface) by a distance, L₁, of 2-4 mm. Alternatively, in some examples, the intermediate portion 208 may extend even farther, for example, by a distance, L₁, of up to 10 mm.

In some examples, the sealing frame 202 can be constructed of a plastically-expandable material, such as stainless steel, biocompatible high-strength alloys (e.g., a cobalt-chromium or a nickel-cobalt-chromium alloys), polymers, or combinations thereof. Alternatively, in some examples, the sealing frame 202 can be constructed of a shape memory material, for example, a nickel titanium alloy such as Nitinol. The shape memory material can be constructed to have an original, pre-deformed shape that corresponds to the desired protruding shape of the sealing frame 202 in the expanded configuration, as shown in FIG. 4A. The sealing frame 202 is then deformed into its compressed configuration shown in FIG. 4B, for example, at a temperature below the transition temperature of the shape memory material. An external force may be used to maintain the sealing frame 202 in the compressed configuration. For example, a sheath can be placed over the entire prosthetic heart valve 200 to keep the sealing frame 202 from expanding. Alternatively or additionally, when valve frame 102 is not formed of a shape memory material, the disposition of the valve frame 102 in its corresponding compressed configuration may provide sufficient resistance to prevent sealing frame 102 from expanding without external actuation (e.g., balloon inflation, mechanical actuation, or otherwise expanding valve frame 102). When the external force is removed and/or the valve frame 102 transitions to the expanded configuration, the sealing frame 202 can be exposed to a temperature greater than its transition temperature. The sealing frame 202 thus remembers and reverts to its original, undeformed state, e.g., the protruding shape illustrated in FIG. 4A. The foreshortening of the valve frame 102 as it transitions from the compressed configuration to the expanded configuration may assist the sealing frame 202 in reverting to its desired pre-deformed shape with the intermediate portion 208 projecting radially outward.

Alternatively or additionally, in some examples, the shape memory material can be constructed to have an original, pre-deformed shape that is intermediate between the sealing frame protruding shape in the expanded configuration of FIG. 4A and the sealing frame non-protruding shape in the compressed configuration of FIG. 4B. For example, the pre-deformed shape of the sealing frame 202 may have an intermediate portion that projects radially outward from an exterior surface of the valve frame by a distance that is less than the distance, L₁, in the fully expanded state. The foreshortening of the valve frame 102 as it transitions to the fully-expanded configuration can thus deform the sealing frame 202 to cause the intermediate portion 208 to further deflect to achieve the desired radial distance L₁.

As shown in FIG. 4D, the prosthetic heart valve 200 further includes an outer skirt 210 that sits radially outward of the sealing frame 202. For example, the sealing frame 202 can cause the outer skirt 210 to have a partial-donut shape around an inflow side of the prosthetic heart valve 200 following deployment of the valve at a desired implantation location (e.g., the native mitral valve in the example of FIG. 4D). The projecting intermediate portion 208 of the sealing frame 202 can thus push the outer skirt 210 radially outward and into contact with the surrounding anatomy (e.g., the native leaflets 44 or other structures of the native mitral annulus in the example of FIG. 4D). The sealing frame 202 in the expanded configuration thus supports the outer skirt 210 and urges it into relatively-stiff anatomical features and/or over relatively-large gaps. Although FIG. 4D shows the prosthetic heart valve 200 (with sealing frame 202 supporting outer skirt 210) implanted at the mitral location with a docking station 152 (also known as a docking device or valve dock), it is also possible to implant the prosthetic heart valve 200 at the mitral location without a docking device or to implant the prosthetic heart valve 200 at any other heart valve location (with or without an appropriate docking device), as noted above.

In practical implementations of an implanted prosthetic heart valve, the shape of the sealing frame 202 in the expanded configuration may be altered due to interaction with surrounding structures (e.g., native tissue). For example, relatively stiff anatomical features may keep the sealing frame 202 from expanding to its full protruding profile shape. Alternatively or additionally, the sealing frame 202 may interact asymmetrically with the surrounding tissue (e.g., due to an irregularly-shaped native annuls, such as at the mitral valve), which may result in different portions of the sealing frame 202 having different cross-sectional profiles. Thus, in determining whether a sealing frame adopts a particular protruding shape and/or follows a desired cross-sectional profile, the prosthetic heart valve 200 with sealing frame 202 may be evaluated in its expanded configuration prior to implantation.

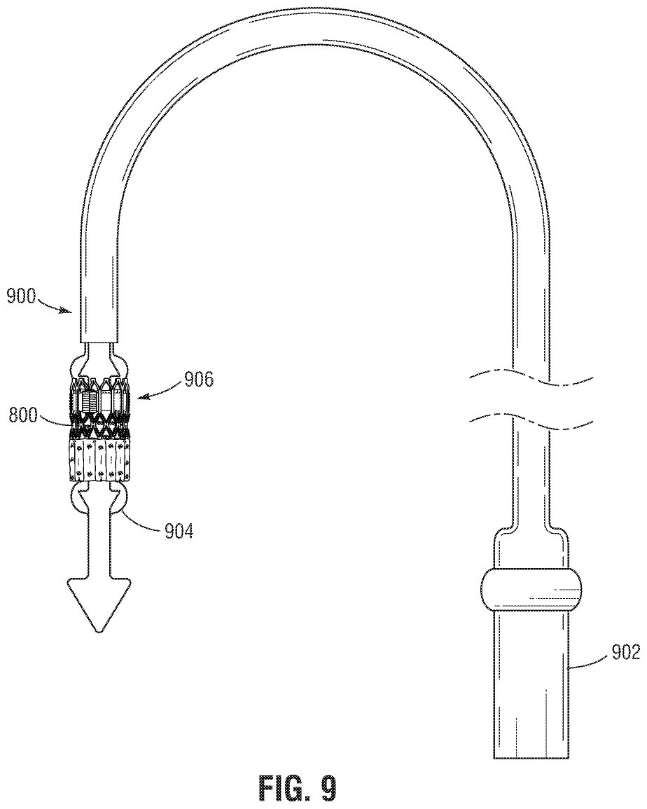

FIGS. 3A-3B and 4C illustrate further details regarding the structure of exemplary prosthetic heart valve 200 that can employ a sealing frame 202. The prosthetic heart valve 200, which can include the annular valve frame 102, the sealing frame 202, and an outer skirt, such as skirt 210, can be crimped on or retained by an implant delivery apparatus in a radially-compressed configuration while the prosthetic heart valve is routed through the anatomy of a patient to the patient's heart, and then expanded to a radially-expanded configuration once the prosthetic heart valve reaches an implantation site within the heart. Prosthetic heart valve 200 can be implanted using any known delivery apparatus, for example, the delivery apparatus illustrated in FIG. 9.

The prosthetic heart valve 200 can include an annular stent or frame 102, which has a first axial end 116 and a second axial end 118. In the depicted example, the first axial end 116 can be an outflow end, and the second axial end 118 can be an inflow end. In some examples, the frame 102, or components thereof (e.g., struts 130, 132, and/or 138), can be made of any of various suitable plastically-expandable materials or self-expanding materials, as known in the art. Plastically-expandable materials that can be used to form the frame 102 can include, but are not limited to, stainless steel, biocompatible high-strength alloys (e.g., a cobalt-chromium or a nickel-cobalt-chromium alloys), polymers, or combinations thereof. In particular examples, frame 102 is made of a nickel-cobalt-chromium-molybdenum alloy, such as MP35N® alloy (SPS Technologies, Jenkintown, Pa.), which is equivalent to UNS R30035 alloy (covered by ASTM F562-02). MP35N® alloy/UNS R30035 alloy comprises 35% nickel, 35% cobalt, 20% chromium, and 10% molybdenum, by weight. Self-expanding materials that can be used to form the frame 102 can include, but are not limited to, nickel titanium alloy (NiTi), such as Nitinol.

When constructed of a plastically-expandable material, the frame 102 (and thus the prosthetic heart valve 200) can be crimped to the radially-compressed configuration on a delivery catheter and then expanded inside a patient by an inflatable balloon or equivalent expansion mechanism. Alternatively, when constructed of a self-expanding material, the frame 102 (and thus the prosthetic heart valve 200) can be crimped to the radially-compressed configuration and restrained in the compressed configuration by insertion into a sheath or equivalent mechanism of a delivery catheter. Once advanced to the implantation site, the prosthetic heart valve can be advanced from the delivery sheath, thereby allowing the prosthetic heart valve to expand to its functional size. Further details of delivery apparatuses that can be used to deliver and implant self-expandable prosthetic valves (including any of the prosthetic valves disclosed herein when the frames are constructed of a self-expandable material such as nitinol) are disclosed in U.S. Pat. Nos. 8,652,202 and 9,867,700, each of which is incorporated herein by reference.

In some examples, struts 130 of the frame 102 are pivotable or bendable relative to each other to permit radial expansion and contraction of the frame 102. For example, the frame 102 can be formed (e.g., via laser cutting, electroforming or physical vapor deposition) from a single piece of material (e.g., a metal tube). In other examples, the frame 102 can be constructed by forming individual components (e.g., the struts and fasteners of the frame) and then mechanically assembling and connecting the individual components together. For example, instead of the strut structure illustrated, the frame can have individual diagonally-extending struts pivotably coupled to one another at one or more pivot joints along the length of each strut, as described in U.S. Pat. Nos. 10,603,165 and 10,806,573, and U.S. Patent Application Publication No. 2018/0344456, all of which are incorporated herein by reference.

The frame 102 can be formed with a plurality of circumferentially-spaced commissure windows 114. A valvular structure 106 can be coupled to the frame 102 at the commissure windows 114. For example, the valvular structure 106 can have a plurality of commissure assemblies 112, each corresponding to a respective one of the commissure windows 114 of the frame 102. In the illustrated example of FIGS. 3A and 4C, the valvular structure 106 comprises three leaflets 110 (e.g., a tricuspid structure), and the commissure windows 114 are equally spaced at 1200 intervals (i.e., 0°, 120°, and 240°) along the circumference of the frame 102. However, other spacings and numbers of commissure windows 114 are also possible according to one or more contemplated examples. For example, in some examples, the valvular structure comprises two leaflets (e.g., a bicuspid structure), and the commissure windows are disposed on opposite sides of the frame (e.g., aligned on a same diameter of the frame).

In the illustrated example of FIG. 3B, each commissure window 114 can have a rectangular construction, with a central opening defined by a pair of side struts 132 (e.g., extending primarily along an axial direction of the frame 102) and a pair of cross-bars (e.g., extending primarily along a circumferential direction of the frame 102 at opposite axial ends of the struts 132). Other shapes and configurations for commissure window 114 are also possible according to one or more contemplated examples. For example, instead of a rectangular opening, the commissure window can define an opening that is square, oval, square-oval, triangular, L-shaped, T-shaped, C-shaped, H-shaped, or any other shape.

Each commissure window 114 can be formed within or part of an open-cell lattice structure formed by axial struts 138 and angled struts 130 (also referred to as rungs). The struts 130, 138 of the frame can form circumferentially-extending rows of open cells. For example, each cell 144 in a first row, which is closest to the inflow end 118 of the frame 102, can be formed by a lower pair of angled struts 130 (which connect together to form an inflow-end apex 154) joined to an upper pair of angled struts 130 (which connect together at second rung junction 162) by a pair of axial struts 138 (which connect to the upper pair of angled struts 130 at a first rung junction 160). Similarly, each cell 150 in a fourth row, which is closest to the outflow end 116 of the frame 102, can be formed by an upper pair of angled struts 130 (which connect together to form an outflow-end apex 156) joined to a lower pair of angled struts 130 (which connect together at third rung junction 164) by pair of axial struts 138 (which connect to the lower pair of angled struts 130 at fourth rung junction 166) or by one of the axial struts 138 and a commissure window side strut 132.

The second row of open cells 146 can be disposed adjacent to and share angled struts with the first row of open cells 144 and the third row of open cells 148. Similarly, the third row of open cells 148 can be disposed adjacent to and share angled struts with the second row of open cells 146 and the fourth row of open cells 150. For example, each cell 146 can be formed by a first pair of angled struts 130 that are connected together at first rung junction 160 and a second pair of angled struts 130 that are connected together at third rung junction 164, with the first and second pairs being connected together via second rung junction 162. For example, each cell 148 can be formed by a third pair of angled struts 130 that are connected together at the second rung junction 162 and a fourth pair of angled struts that are connected together at either the fourth rung junction 166 or a junction with a commissure window 114, with the third and fourth pairs being connected together via the third rung junction 164.

In some examples, one or more of the inflow-end apices 154 (or portions of struts 130 forming apices 154) can serve as attachment points for the sealing frame 202, for example, second axial end 206. For example, in some examples, apices formed by struts of the sealing frame 202 at the second axial end 206 can be aligned with apices 154 of the valve frame 102 in a one-to-one correspondence, and the apices of the sealing frame and the valve frame 102 can be coupled together via one or more sutures, adhesive, welding, or any other suitable attachment means. Alternatively, in some examples, apices formed by struts of the sealing frame 202 at the second axial end 206 can be aligned with some of the apices 154, such that only every other apex 154 of the valve frame 102 is aligned with a corresponding apex of the sealing frame 202. Again, the aligned apices of the valve frame 102 and the sealing frame 202 can be coupled together via one or more sutures, adhesive, welding, or any other suitable attachment means.

In some examples, one or more of the rung junctions 160-166 (or portions of angled struts 130 and/or axial struts 138 forming such rung junctions) can serve as attachment points for the sealing frame 202, for example, first axial end 204. For example, apices formed by struts of the sealing frame 202 at the first axial end 204 can be aligned with all of the first rung junctions 160 (e.g., in a one-to-one correspondence) or only some of the first rung junctions 160 (e.g., every other rung junction) of the valve frame 102. In another example, apices formed by struts of the sealing frame 202 at the first axial end 204 can be aligned with all of the second rung junctions 162 (e.g., in a one-to-one correspondence) or only some of the second rung junctions 162 (e.g., every other rung junction) of the valve frame 102. In a further example, apices formed by struts of the sealing frame 202 at the first axial end 204 can be aligned with all of the third rung junctions 164 (e.g., in a one-to-one correspondence) or only some of the third rung junctions 164 (e.g., every other rung junction) of the valve frame 102. In still another example, apices formed by struts of the sealing frame 202 at the first axial end 204 can be aligned with all of the fourth rung junctions 166 and/or any junction with commissure window 114 (e.g., in a one-to-one correspondence), or only some of the fourth rung junctions 166 and/or junction with commissure window 114 (e.g., every other rung junction) of the valve frame 102. In any of the above noted examples, the sealing frame apices aligned with valve frame rung junctions can be coupled thereto via one or more sutures, adhesive, welding, or any other suitable attachment means.

The valvular structure 106 can be configured to allow blood flow through the frame 102 in only one direction, for example, to regulate the flow of blood through the prosthetic heart valve 200 from the inflow end 118 to the outflow end 116. Thus, the leaflets 110 of the valvular structure 106 can transition between an open configuration, where blood flows through the valve 200 via a flow channel formed by the leaflets 110, and a closed configuration, where the leaflets 110 occlude blood flow through the valve 200. The leaflets 110 can be made from in whole or part, biological materials, bio-compatible synthetic materials, or other such materials. Suitable biological materials can include, for example, bovine pericardium (or pericardium from other sources). Each commissure assembly 112 formed by paired tabs of adjacent leaflets 110 can be inserted through the opening of the corresponding commissure window 114 and attached to the window 114, for example, via one or more sutures. Further details regarding valvular structure construction and coupling to the valve frame are disclosed in U.S. Pat. No. 9,393,110, which is incorporated herein by reference.

As shown in FIGS. 3A and 4C, the prosthetic heart valve 200 can also include an inner skirt 108 mounted on an inner surface of the frame 102. The inner skirt 108 can be a circumferential inner skirt that spans an entire circumference of the inner surface of the frame 102. The inner skirt 108 can be formed of any of various suitable biocompatible materials, including any of various synthetic materials (e.g., polyethylene terephthalate (PET)) or natural tissue (e.g., pericardial tissue). The inner skirt 108 can function as a sealing member to prevent or decrease PVL (e.g., when the valve is placed at the implantation site) and as an attachment surface to anchor a portion of the leaflets 110 to the frame 102. For example, the cusp edge portions of leaflets 110 can be sutured to the inner skirt 108, which in turn can be sutured to selected struts 130 of the frame 102. Alternatively or additionally, the inner skirt 108 can be coupled to the frame 102 and/or to the leaflets 110 via adhesive, welding, and/or any other means for attachment. Further details regarding frame construction, inner skirts, techniques for assembling leaflets to the inner skirt, and techniques for assembling skirts to the frame are disclosed in U.S. Pat. No. 9,393,110, U.S. Patent Application Publication Nos. 2019/0192296 and 2019/0365530, and International Publication Nos. WO/2020/159783 and WO/2020/198273, each of which is incorporated herein by reference.

As shown in FIG. 4C, the outer skirt 210 can be mounted over an exterior of the valve frame 102. The sealing frame 202 can be disposed along the radial direction between the outer skirt 210 and the valve frame 102, such that the outer skirt 210 covers the sealing frame 202. The outer skirt 210 can span an entire circumference of the exterior of the valve 102 and the sealing frame 202. The outer skirt 210, supported internally by the sealing frame 202, can function as an improved sealing member by closing any gaps between the valve frame 102 and surrounding structures (e.g., the tissue of the native valve annulus), thereby helping to reduce PVL past the prosthetic valve 200. The outer skirt 210 can be formed of any of various suitable biocompatible materials, including any of various synthetic materials or natural tissue (e.g., pericardial tissue). For example, the outer skirt can be formed of polyethylene terephthalate (PET), polyurethane (PU), a matrix of PU and polycarbonate (PC), expanded polytetrafluoroethylene (ePTFE), composite materials, or any combination thereof.

In some examples, the outer skirt 210 extends along an axial direction of the frame 102 from a location at or just beyond an attachment point of the second axial end 206 of the sealing frame 202 to a location at or just beyond an attachment point of the first axial end 204 of the sealing frame 202 to the valve frame 102. For example, the outer skirt 210 can extend along an axial direction of the valve frame 102 from a location at or around the inflow end 118 of the frame (e.g., wrapping around the inflow end 118 to attach to the inner skirt 108 on a radially-inner side of the valve frame 102) to a location intermediate between the inflow end 118 and commissures 112, as illustrated in FIG. 4C. Alternatively, in some examples, the outer skirt may extend substantially along an entire height of the valve frame 102 to, in effect, cover an entire radially-outer surface of the valve frame 102. In such configurations, the outer skirt may be formed of materials that contribute to improved compressibility or compliance, which, in combination with the support of the underlying sealing frame 202, can enhance the ability of the outer skirt to effectively seal any gaps between the valve frame 102 and surrounding structures. For example, the outer skirt can be made of non-woven fabric (e.g., felt), non-woven fibers (e.g., non-woven cotton fibers), porous or spongey materials (e.g., any of a variety of compliant polymeric foam materials), or woven or knitted fabrics (e.g., woven or knitted PET). Further details regarding prosthetic heart valves with outer skirts that completely cover the valve frame, which prosthetic valves can benefit from the disclosed sealing frame, are described in U.S. Patent Application Publication Nos. 2019/0374337, 2019/0192296, and 2019/0046314, each of which is incorporated herein by reference.

In some examples, the outer skirt 210 can be directly coupled to the valve frame 102. For example, extension portions of the outer skirt 210 closest to the outflow end 116 of the valve frame 102 can be wrapped at least partially around angled struts 130 and second rung junction 162 and attached thereto via one or more sutures, adhesive, welding, or any other suitable attachment means. Portions of the outer skirt 210 closest to the inflow end 118 can be wrapped around the inflow end 118 and attached to the inner skirt 108 on a radially-inner side of the valve frame 102 via one or more sutures, adhesive, welding, or any other suitable attachment means. Alternatively or additionally, in some examples, the outer skirt 210 can be indirectly coupled to the valve frame 102, for example, by being directly coupled to the sealing frame 202. For example, the outer skirt 210 can be attached to portions of the struts at the first axial end 204 and/or to portions of the struts at the second axial end 206 of the sealing frame 202 via one or more sutures, adhesive, welding, or any other suitable attachment means. In some examples, an intermediate portion of the outer skirt 210 (e.g., between axial ends of the outer skirt 210 that are otherwise directly or indirectly coupled to the valve frame 102) remains unattached and free to move, for example, to accommodate the transition of the underlying sealing frame 202 between its compressed and expanded configurations.

Although the above discussion describes a particular configuration for prosthetic heart valve 200, examples of the disclosed subject matter are not limited thereto. Rather, the combination of outer skirt and sealing frame 202 can be applied to many other prosthetic heart valve configurations as well. For example, in some examples, the prosthetic valve includes one or more actuators coupled to the frame to cause transition of the valve between crimped and expanded configurations, and/or one or more locking mechanisms that maintains a shape of the frame after expansion or contraction. In addition to or in place of commissure windows provided in the lattice structure of the frame, at least one of the actuators or locking mechanisms can include a commissure window formed therein and can be used to mount a commissure of the valvular assembly thereto. Further details of valve frames employing actuators and delivery apparatuses for actuating the actuators can be found in U.S. Pat. Nos. 10,603,165 and 10,806,573, U.S. Patent Application Publication No. 2018/0325665, and International Publication No. WO/2020/102487, all of which are incorporated herein by reference.

Moreover, although the above discussion has focused on prosthetic heart valves that are substantially or generally cylindrical in shape (e.g., having an annular valve frame), the disclosed subject matter is not limited thereto. Indeed, prosthetic heart valves having non-cylindrical geometries, such as hour-glass, tapered or frustoconical, may also benefit from the use of a sealing frame to urge an outer skirt into contact with surrounding structures (of the native anatomy or an implanted device, such as a docking station or previously implanted prosthetic valve) to close off gaps that might have otherwise contributed to PVL. Further details of non-cylindrical valve frames, which can benefit from the disclosed sealing frames, are described in U.S. Pat. No. 8,652,202, U.S. Patent Application Publication No. 2020/0188099, and International Publication No. WO/2020/081893, all of which are incorporated herein by reference.

As noted above, in some examples, a sealing frame can be formed by a network of struts that connect together to form closed cells (e.g., closed along the axial direction with respect to both the inflow and outflow ends of the valve). FIG. 5A illustrates an example of such a sealing frame 302 with closed cells 311 in an expanded configuration. FIGS. 5B and 5D illustrate an exemplary arrangement of the expanded sealing frame 302 with respect to the valve frame 102 and outer skirt 330. FIG. 5C shows an exemplary arrangement of and attachment between the sealing frame 302 and outer skirt 330.

In the illustrated example of FIG. 5A, the sealing frame 302 is formed by a plurality of elementary units 310 arrayed around a circumference of the valve frame and coupled together. Each elementary unit 310 can have a first apex 322 at one axial end and a second apex 324 at an opposite axial end. The first apices 322 of the elementary units 310 can define a first axial end portion 304 that is coupled to the valve frame 102 (e.g., at a location remote from the inflow end 118), and the second apices 324 of the elementary units 310 can define a second axial end portion 306 that is coupled to the valve frame 102 (e.g., at or near the inflow end 118). In the illustrated example of FIG. 5A, each of the first apices 322 has a first eyelet 326, and each of the second apices 324 has a second eyelet 328. One or more sutures (e.g., sutures 338 in FIG. 5D) can be passed through the eyelets 326, 328 in order to couple the respective apices 322, 324 to portions of the valve frame 102. Alternatively, instead of providing eyelets 326, 328, sutures can be wrapped around the respective apices 322, 324.

Each elementary unit 310 can have a first upper angled strut portion 312a, a second upper angled strut portion 312b, a first lower angled strut portion 314a, and a second lower angled strut portion 314b. The first and second upper angled strut portions 312a, 312b are connected to each other via first apex 322, and the first and second lower angled strut portions 314a, 314b are connected to each other via second apex 324. The upper angled strut portions 312a, 312b are connected to the lower angled strut portions 314a, 314b by a first coupling portion 320a and a second coupling portion 320b. Each coupling portion 320a, 320b is also shared with adjacent elementary units 310 to form the sealing frame 302 as a continuous array of elementary units 310 that surrounds an outer circumference of the valve frame 102. In some examples, the coupling portions 320a, 320b of the elementary units 310 can be considered an intermediate portion 308 that projects radially outward from the valve frame, as illustrated in FIG. 5B, when the sealing frame is in the expanded configuration.

Angled strut portions 312, 314 and coupling portions 320 together define a closed cell 311. In the expanded configuration of the sealing frame 302 shown in FIG. 5A, the coupling portions 320a, 320b are spaced apart from each other along the circumferential direction. However, when the sealing frame 302 transitions to the compressed configuration, the apices 322, 324 move away from each other with respect to the axial direction of the valve frame 102 and the coupling portions 320a, 320b move toward each other along the circumferential direction. The final compressed configuration may be a state where the coupling portions 320 are in contact with each other (or almost touching) and/or where each angled strut portion 312, 314 is substantially parallel to an axial direction of the valve frame 102. Alternatively or additionally, the final compressed configuration may be a state where the first axial end portion 304 and the second axial end portion 306 have an inner diameter that substantially matches an outer diameter of the valve frame 102 in its compressed configuration. With the sealing frame 302 in its final compressed configuration, the radial projection of intermediate portion 308 can be eliminated, or at least reduced, due to the positioning of the angled struts 312, 314 forming cell 311 of each elementary unit 310. Thus, the cell configuration of the sealing frame 302 can allow it to adopt a low profile (e.g., minimal diameter) in the compressed configuration for transcatheter delivery to the implantation site, and to adopt a radially-projecting profile in the expanded configuration once delivered to the implantation site.

In the illustrated example of FIG. 5A, each elementary unit 310 is symmetric with respect to an axially-extending centerline between the first apex 322 and the second apex 324, and each elementary unit 310 is symmetric with respect to a circumferentially-extending centerline between first coupling portion 320a and second coupling portion 320b. However, in some examples, each elementary unit 310 may be asymmetrical with respect to one or more directions, such as a line joining first apex 322 and second apex 324, or a line joining first coupling portion 320a and second coupling portion 320b. For example, the upper angled strut portions 312 may have a greater height (along the axial direction of the valve frame) than that of the lower angled strut portions 314, such that the coupling portions 320 (and thus protruding intermediate portion 308) are disposed closer to an inflow end 118 of the valve frame 102.

In some examples, the sealing frame 302 is constructed of a shape memory material, such as a nickel titanium alloy (e.g., Nitinol). The sealing frame 302 can be constructed such that its original pre-deformed shape has the intermediate portion 308 projecting radially outward (e.g., as compared to the radial locations of the first axial end portion 304, the second axial end portion 306, and/or a radially-outer circumferential surface of the valve frame 102). The sealing frame 302 can then transition to its compressed configuration, for example, by radially compressing the intermediate portion 308 and/or by displacing the first axial end portion 304 and the second axial portion 306 away from each other. By exposing the sealing frame 302 in the compressed configuration to a temperature in excess of its transition temperature and after release from any external restriction (e.g., a sheath of the delivery apparatus), the sealing frame 302 automatically reverts to is original pre-deformed shape, e.g., the profile for the expanded configuration illustrated in FIG. 5A. The transition temperature of the shape memory material may be tailored by appropriate selection of the material composition. In some examples, the shape memory alloy has a transition temperature (e.g., 30° C.) that is less than a normal body temperature of the patient (e.g., 37° C.).

The sealing frame 302 can be constructed by any number of fabrication techniques. For example, in some examples, the sealing frame 302 is constructed by laser cutting a shape memory material tube to form the respective upper angled strut portions 312, lower angled strut portions 314, coupling portions 320, first apices 322, and second apices 324. Alternatively or additionally, in some examples, the sealing frame 302 can be constructed by laser welding of individual shape memory material wires together, for example, where a first wire forms the first upper angled strut portion 312a, the second coupling portion 320b, and the second lower angled strut portion 314b, and a second wire forms the second upper angled strut portion 312b, the first coupling portion 320a, and the first lower angled strut portion 314a. In either example, laser cutting or mechanical machining can be used to form the first eyelets 326 and the second eyelets 328. In some examples, after forming the interconnected strut structure, intermediate portion 308 of the sealing frame 302 can be modified such that it projects radially outward, as illustrated in FIG. 5A. In some examples, the intermediate portion 308 can be displaced radially outward from the first and second axial end portions 304, 306 (e.g., by using a preformed or inflatable mandrel) while the sealing frame 302 is held at a temperature in excess of the transition temperature. Alternatively or additionally, the first and second axial portions 304, 306 can be displaced radially inward from the intermediate portion 308 (e.g., by using a mandrel, rollers, and/or a lathe). The sealing frame 302 can then be cooled to a temperature below the transition temperature to set its current shape as the original pre-deformed shape. Alternatively or additionally, individual wires can be bent above their transition temperatures to have appropriate protruding portions that correspond to the desired intermediate portion 308. The wires can then be coupled together, for example, by laser welding, to form the interconnected strut structure for sealing frame 302. Deformation of the sealing frame 302 while below the transition temperature (e.g., in transitioning to the compressed configuration) can be effectively undone by heating to a temperature about the transition temperature, whereby the sealing frame 302 automatically reverts to its original pre-deformed shape. Further details regarding shape memory material fabrication techniques, which can be used to form sealing frame 302, can be found in U.S. Pat. Nos. 5,540,712 and 8,187,396, each of which is incorporated herein by reference.

In some examples, the outer skirt may be coupled to the sealing frame prior to attachment of the sealing frame to the valve frame. For example, one or more sutures may be used to attach the outer skirt to strut portions of the sealing frame. In some examples, the sutures can follow a perimeter of each elementary unit cell and wrap around the individual strut portions defining the elementary unit cell. Alternatively, the outer skirt can be attached to individual strut portions via respective sutures close to or at the axial ends defined by the apices, without any direct coupling between the sealing frame and the portion of the outer skirt corresponding to the intermediate portion defined by the coupling portions. For example, outer skirt 330 is attached to sealing frame 302 via a plurality of sutures 334 that wrap around lower angled strut portions 314 near second apices 324 and a plurality of sutures 334 that wrap around upper angled strut portions 312 near first apices 322, as shown in FIG. 5C. However, a central portion of the outer skirt 330 corresponding to coupling portion 320 can remain uncoupled, for example, such that the outer skirt 330 can accommodate changes in shape of the sealing frame as it transitions between expanded and compressed configurations.

The outer skirt 330 can have a first edge portion 336 positioned closest to the inflow end 118 of the valve frame 102. In some examples, the first edge portion 336 can be separately coupled to the valve frame 102. For example, the first edge portion 336 can be wrapped around the inflow end 118 of the valve frame 102 to be attached via one or more sutures to a radially-inner circumferential surface of the valve frame 102, or to an inner skirt (e.g., inner skirt 108) on the radially-inner circumferential surface of the valve frame 102. In some examples, opposite to the first edge portion 336 along the axial direction, the outer skirt 330 can have a second edge portion 332 positioned closest to the outflow end 116 of the valve frame 102. In some examples, the second edge portion 332 is a patterned edge portion with portions 332a that project axially toward the outflow end 116 of the valve frame 102 (e.g., to have an undulating pattern). For example, each axially-projecting portion 332a can be aligned with a respective one of the first apices 322, as shown in FIG. 5C. Alternatively, in some examples, the second edge portion of the outer skirt may follow a substantially straight edge (e.g., with the edge extending around the circumference of the valve frame 102 at a substantially constant distance with respect to the inflow end 118 or the outflow end 116).

In some examples, the sealing frame 302 can be constructed and attached to the valve frame 102 such that each elementary unit 310 corresponds to a cell of the valve frame 102. In such configurations, the first apex 322 can be attached to a rung junction of the valve frame cell, and the second apex 324 can be attached to an inflow-end apex of the valve frame. For example, as shown in FIG. 5D, the elementary units of sealing frame 302 correspond to the first row of cells 144 of the valve frame 102 at the inflow end 118 thereof. Each first apex 322 of the sealing frame 302 is aligned with a respect second rung junction 162 of the valve frame 102. One or more sutures 338 can pass through eyelets 326 of the first apices 322 and wrap around the second rung junctions 162 to securely couple the first axial end portion 304 of the sealing frame 302 to the valve frame 102. One or more sutures 338 can pass through eyelets 328 of the second apices 324 and wrap around the inflow-end apices 154 to securely couple the second axial end portion 306 of the sealing frame 302 to the valve frame 102.

In some examples, once the sealing frame 302 with outer skirt 330 coupled thereto is attached to the valve frame 102, the outer skirt 330 can then be separately coupled to the valve frame 102. For example, as described above, part of the outer skirt can be wrapped around the inflow end 118 of the valve frame 102 to be coupled at a radially-inner side of the valve frame 102. Alternatively or additionally, part of the outer skirt can be separately coupled at a radially-outer side of the valve frame, for example, by suturing the first edge portion to angled struts 130 extending from inflow apices 154 of the valve frame 102. It should be noted that the illustration in FIG. 5D shows the outer skirt 330 in dashed outline only to avoid obscuring the underlying sealing and valve frame structures. FIG. 5D thus does not show coupling of the outer skirt to the sealing frame 302 (e.g., via sutures 334) or to the valve frame 102.

In FIG. 5D, the outer skirt 330 is coupled to the sealing frame 302. Alternatively, in some examples, the outer skirt can be coupled to the valve frame 102 instead of the sealing frame 302, for example, as illustrated in FIG. 5E. The sealing frame 302 can be coupled to valve frame 102 in a manner similar to that described above with respect to FIG. 5D. Once the sealing frame 302 is secured to the valve frame 102, an outer skirt 350 can then be draped over the sealing frame 302. Similar to the outer skirt 330 illustrated in FIGS. 5C-5D, outer skirt 350 can have a first edge portion positioned closest to the inflow edge 118 of the valve frame 102 and an opposite second edge portion 352 positioned closest to the outflow edge 116 of the valve frame 102. The first edge portion of the outer skirt 350 can be wrapped around the inflow end 118 of the valve frame 102 for attachment on a radially-inner side of the valve frame 102 (e.g., to inner skirt 108). As shown in FIG. 5E, in some examples, the second edge portion 352 can be patterned with portions 352a that project axially toward the outflow end 116 of the valve frame 102. However, in contrast to the configuration of outer skirt 330 in FIGS. 5C-5D, the outer skirt 350 extends along the axial direction of the valve frame 102 beyond the first apices 322 of the sealing frame 302. In addition, instead of being aligned with the first apices 322, axially-projecting portions 352a can be offset from the first apices 322 along a circumferential direction of the valve frame 102. The projection portions 352a can thus extend to and be aligned with respective third rung junctions 164, where one or more sutures 354 can be used to attach the projecting portions 352a to the valve frame 102 (e.g., angled struts 130 extending from third rung junction 164). It should be noted that the illustration in FIG. 5E shows the outer skirt 350 in dashed outline only to avoid obscuring the underlying sealing and valve frame structures. Some sutures 354 that attach the second end portion 352 of the outer skirt 350 to the valve frame 102 are also shown, although additional sutures and/or different suture configurations are also possible.

In the examples illustrated in FIGS. 5D and 5E, the sealing frame 302 is constructed such that the cell 311 of each elementary unit 310 extends over a single row of valve frame cells, e.g., the first row of cells 144 at the inflow end 118 of the valve frame 102. Alternatively, in some examples, the sealing frame can extend over multiple rows of cells of valve frame 102. For example, FIG. 5F shows a sealing frame 362 attached to a valve frame 102. Similar to sealing frame 302, sealing frame 362 can be formed by a plurality of elementary units arrayed around the circumferential direction and connected together. Each elementary unit can have a pair of upper angled strut portions 364 extending from a first apex 368, a pair of lower angled strut portions 366 extending from a second apex 370, and a pair of coupling portions 378 connecting the upper angled strut portions to the lower angled strut portions and connecting adjacent elementary units together. Each first apex 368 can be aligned with and attached to a respective junction (e.g., fourth rung junction 166) of the valve frame 102 by one or more sutures 376, and each second apex 370 can be aligned with and attached to a respective inflow apex 154 of the valve frame 102 by one or more sutures 376. However, in contrast to the sealing frame 302, the sealing frame 362 extends along the axial direction a greater distance such that the sealing frame 362 extends over multiple rows of cells of the valve frame 102, e.g., first row of cells 144, second row of cells 146, and third row of cells 148.

Outer skirt 372 can be attached to sealing frame 362 via a plurality of sutures in a manner similar to that described above with respect to FIG. 5C. The outer skirt 372 can have a first edge portion positioned closest to the inflow end 118 of the valve frame 102. In some examples, the first edge portion of outer skirt 372 can be separately coupled to the valve frame 102. For example, the first edge portion can be wrapped around the inflow end 118 of the valve frame 102 to be attached via one or more sutures to a radially-inner circumferential surface of the valve frame 102, or to an inner skirt (e.g., inner skirt 108) on the radially-inner circumferential surface of the valve frame 102. In some examples, the outer skirt 372 can have a second edge portion 374 positioned closest to the outflow end 116 of the valve frame 102. In some examples, the second edge portion 374 is a patterned edge portion with portions 374a that project axially toward the outflow end 116 of the valve frame 102. For example, each axially-projecting portion 374a can be aligned with a respective one of the first apices 368, as shown in FIG. 5F. Alternatively, in some examples, the second edge portion of the outer skirt may follow a substantially straight edge (e.g., with the edge extending around the circumference of the valve frame 102 at a substantially constant distance with respect to the inflow end 118 or the outflow end 116).

In some examples, once the sealing frame 362 with outer skirt 372 coupled thereto is attached to the valve frame 102, the outer skirt 372 can then be separately coupled to the valve frame 102. For example, as described above, part of the outer skirt can be wrapped around the inflow end 118 of the valve frame 102 to be coupled at a radially-inner side of the valve frame 102. Alternatively or additionally, part of the outer skirt can be separately coupled at a radially-outer side of the valve frame, for example, by suturing the first edge portion to angled struts 130 extending from inflow apices 154 of the valve frame 102. It should be noted that the illustration in FIG. 5F shows the outer skirt 372 in dashed outline only to avoid obscuring the underlying sealing and valve frame structures. FIG. 5F thus does not show coupling of the outer skirt 372 to the sealing frame 362 (e.g., via sutures 334) or to the valve frame 102.

In the examples illustrated in FIGS. 5D-5F, the second apices of the sealing frame (e.g., apices at the inflow end) have a one-to-one correspondence with the inflow apices 154 of the valve frame 102, and the first apices of the sealing frame (e.g., apices at the outflow end) have a one-to-one correspondence with junctions (e.g., second rung junction 162 or fourth rung junction 166). Alternatively, in some examples, the correspondence between the apices of the sealing frame and the apices/junctions of the valve frame 102 can be other than one-to-one. For example, FIG. 5G shows a sealing frame 380 attached to the valve frame 102 at every other inflow apex 154 and at every other fourth rung junction 166. Similar to sealing frame 362 in FIG. 5F, sealing frame 380 can be formed by a plurality of elementary units arrayed around the circumferential direction and connected together. Each elementary unit can have a pair of upper angled strut portions 382 extending from a first apex 386, a pair of lower angled strut portions 384 extending from a second apex 388, and a pair of coupling portions 390 connecting the upper angled strut portions to the lower angled strut portions and connecting adjacent elementary units together. Each first apex 386 can be aligned with and attached to a respective junction (e.g., fourth rung junction 166) of the valve frame 102 by one or more sutures 392, and each second apex 388 can be aligned with and attached to a respective inflow apex 154a of the valve frame 102 by one or more sutures 392. However, in contrast to the sealing frame 362, each elementary unit of sealing frame 380 extends farther along the circumferential direction, such that the sealing frame 380 is attached at every other inflow apex (e.g., attached at apex 154a but not at apex 154b) and at every other fourth rung junction 166.

Outer skirt 394 can be attached to sealing frame 380 via a plurality of sutures in a manner similar to that described above with respect to FIG. 5C. The outer skirt 394 can have a first edge portion positioned closest to the inflow end 118 of the valve frame 102. In some examples, the first edge portion of outer skirt 394 can be separately coupled to the valve frame 102. For example, the first edge portion can be wrapped around the inflow end 118 of the valve frame 102 to be attached via one or more sutures to a radially-inner circumferential surface of the valve frame 102, or to an inner skirt (e.g., inner skirt 108) on the radially-inner circumferential surface of the valve frame 102. In some examples, the outer skirt 394 can have a second edge portion 396 positioned closest to the outflow end 116 of the valve frame 102. In some examples, the second edge portion 396 is a patterned edge portion with portions 396a that project axially toward the outflow end 116 of the valve frame 102. For example, each axially-projecting portion 396a can be aligned with a respective one of the first apices 386, as shown in FIG. 5G. Alternatively, in some examples, the second edge portion of the outer skirt may follow a substantially straight edge (e.g., with the edge extending around the circumference of the valve frame 102 at a substantially constant distance with respect to the inflow end 118 or the outflow end 116).

In some examples, once the sealing frame 380 with outer skirt 394 coupled thereto is attached to the valve frame 102, the outer skirt 394 can then be separately coupled to the valve frame 102. For example, as described above, part of the outer skirt can be wrapped around the inflow end 118 of the valve frame 102 to be coupled at a radially-inner side of the valve frame 102. Alternatively or additionally, part of the outer skirt can be separately coupled at a radially-outer side of the valve frame, for example, by suturing the first edge portion to angled struts 130 extending from inflow apices 154a, 154b of the valve frame 102. It should be noted that the illustration in FIG. 5G shows the outer skirt 394 in dashed outline only to avoid obscuring the underlying sealing and valve frame structures. FIG. 5G thus does not show coupling of the outer skirt 394 to the sealing frame 380 (e.g., via sutures 334) or to the valve frame 102.

Although specific examples are discussed above, other examples are also possible in one or more implementations. For example, FIG. 5G illustrates sealing frame 380 spanning multiple rows of cells of the valve frame 102 (e.g., cells 144, 146, and 148). However, it also possible for the sealing frame of FIG. 5G to be shortened along the axial direction and thereby span fewer rows of cells. For example, the sealing frame illustrated in FIG. 5G can be modified to cover a single row of cells, such as the first row of cells 144 of the valve frame 102. In such a configuration, the second apices of the sealing frame can continue to be attached to every other inflow apex 154a of the valve frame 102, and the first apices of the sealing frame can instead be attached to every other second rung junction 162 of the valve frame. In another example, FIGS. 5D and 5F-5G describe the outer skirt being directly attached to the sealing frame prior to attachment of the sealing frame to the valve frame. However, it is also possible for the sealing frame to be attached to the valve frame first, and then attach the outer skirt directly to the sealing frame. Alternatively or additionally, the outer skirt in any of FIGS. 5D and 5F-5G can be modified to attach directly to the valve frame, for example, in a manner similar to that described above with respect to FIG. 5E.

It should also be noted that the illustrations in FIGS. 5C-5G show the sealing frame, outer skirt, and valve frame in a flat planar layout for convenience only. In particular implementations, the sealing frame will have the three-dimensional profile when in the expanded configuration (e.g., the profile illustrated in FIG. 5A), and the valve frame 102 will have the annular configuration illustrated in FIGS. 3B and 4C. The coupling of the outer skirt to the sealing frame may occur with the sealing frame having such a three-dimensional profile, and the coupling of the sealing frame to the annular valve frame may occur with the sealing frame having such a three-dimensional profile.

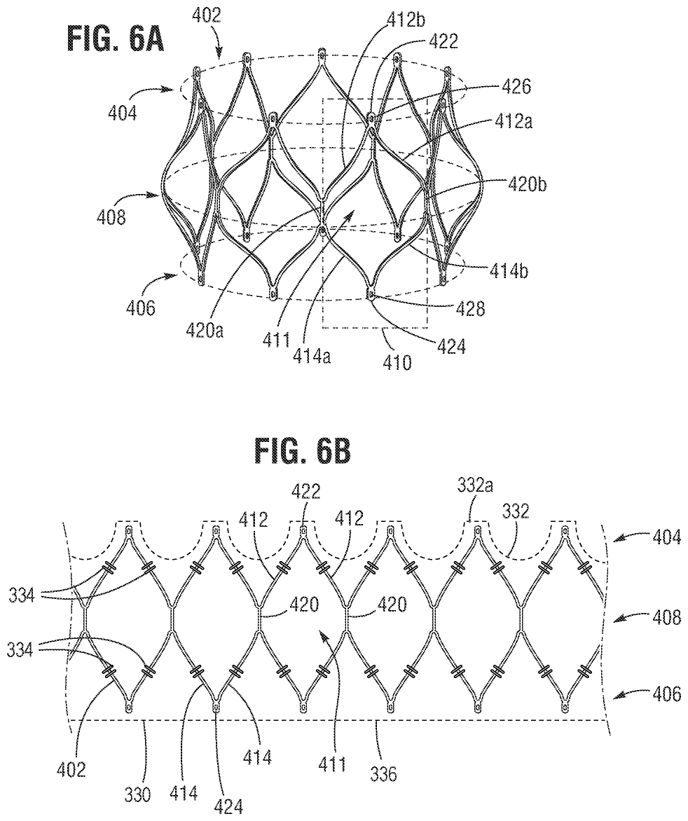

FIG. 6A illustrates another example of a sealing frame 402 with closed cells 411 in an expanded configuration. FIG. 6B shows an exemplary arrangement of and attachment between the sealing frame 402 and outer skirt 430. In the illustrated example of FIG. 6A, the sealing frame 402 is formed by a plurality of elementary units 410 arrayed around a circumference of the valve frame and coupled together. Each elementary unit 410 can have a first apex 422 at one axial end and a second apex 424 at an opposite axial end. The first apices 422 of the elementary units 410 can define a first axial end portion 404 that is coupled to the valve frame 102 (e.g., at a location remote from the inflow end 118), and the second apices 424 of the elementary units 410 can define a second axial end portion 406 that is coupled to the valve frame 102 (e.g., at or near the inflow end 118). In the illustrated example of FIG. 6A, each of the first apices 422 has a first eyelet 426, and each of the second apices 424 has a second eyelet 428. One or more sutures can be passed through the eyelets 426, 428 in order to couple the respective apices 422, 424 to portions of the valve frame 102. Alternatively, instead of providing eyelets 426, 428, sutures can be wrapped around the respective apices 422, 424.

Each elementary unit 410 can have a first upper angled strut 412a, a second upper angled strut 412b, a first lower angled strut 414a, and a second lower angled strut 414b. The first and second upper angled struts 412a, 412b are connected to each other via first apex 422, and the first and second lower angled struts 414a, 414b are connected to each other via second apex 424. The upper angled struts 412a, 412b are connected to the lower angled struts 414a, 414b by a first longitudinal strut 420a and a second longitudinal strut 420b. Each longitudinal strut 420a, 420b is also shared with adjacent elementary units 410 to form the sealing frame 402 as a continuous array of elementary units 410 that surrounds an outer circumference of the valve frame 102. In some examples, the longitudinal struts 420a, 420b of the elementary units 410 can be considered an intermediate portion 408 that projects radially outward from the valve frame, as illustrated in FIG. 6A, when the sealing frame is in the expanded configuration. In the expanded configuration, the longitudinal struts 420a, 420b may thus have a curved shape, such as C-shape, in side view.

Angled struts 412, 414 and longitudinal struts 420 together define a closed cell 411. In the expanded configuration of the sealing frame 402 shown in FIG. 6A, the longitudinal struts 420 are spaced apart from each other along the circumferential direction. However, when the sealing frame 402 transitions to the compressed configuration, the apices 422, 424 move away from each other with respect to the axial direction of the valve frame 102 and the longitudinal struts 420 move toward each other along the circumferential direction. The final compressed configuration may be a state where the longitudinal struts 420 are in contact with each other (or almost touching) and/or where each angled strut 412, 414 is substantially parallel to an axial direction of the valve frame 102. Alternatively or additionally, the final compressed configuration may be a state where the first axial end portion 404 and the second axial end portion 406 have an inner diameter that substantially matches an outer diameter of the valve frame 102 in its compressed configuration. With sealing frame 402 in its final compressed configuration, the radial projection of intermediate portion 408 can be eliminated, or at least reduced, due to the positioning of the angled struts 412, 414 forming cell 411 of each elementary unit 410. Thus, the cell configuration of the sealing frame 402 can allow it to adopt a low profile (e.g., minimal diameter) in the compressed configuration for transcatheter delivery to the implantation site, and to adopt a radially-projecting profile in the expanded configuration once delivered to the implantation site.

In the illustrated example of FIG. 6A, each elementary unit 410 is symmetric with respect to an axially-extending centerline between the first apex 422 and the second apex 424, and each elementary unit 410 is symmetric with respect to a circumferentially-extending centerline between a center of the first longitudinal strut 420a and a center of the second longitudinal strut 420b. However, in some examples, each elementary unit 410 may be asymmetrical with respect to one or more directions, such as a line joining first apex 422 and second apex 424, or a line joining centers of the first longitudinal strut 420a and the second longitudinal strut 420b. For example, the upper angled struts 412 may have a greater height (along the axial direction of the valve frame 102) than that of the lower angled struts 414, such that the longitudinal struts 420 (and thus protruding intermediate portion 408) are disposed closer to an inflow end 118 of the valve frame 102.

The sealing frame 402 is thus structurally similar to the sealing frame illustrated in FIGS. 5A-5G, but with longitudinally-extending struts 420 replacing coupling portions 320. In some examples, sealing frame 402 can be constructed of a shape memory material, such as nickel titanium alloy (e.g., Nitinol). Accordingly, in some examples, sealing frame 402 can be constructed in a manner similar to that describe above with respect to sealing frame 302, and/or sealing frame 402 can be arranged with respect to valve frame 102 and attached thereto in a manner similar to any of those discussed above with respect to FIGS. 5C-5G. Moreover, in some examples, the corresponding outer skirt can be constructed and coupled to the sealing frame 402 (or directly to the valve frame 102) in a manner similar to any of those discussed above with respect to FIGS. 5C-5G.

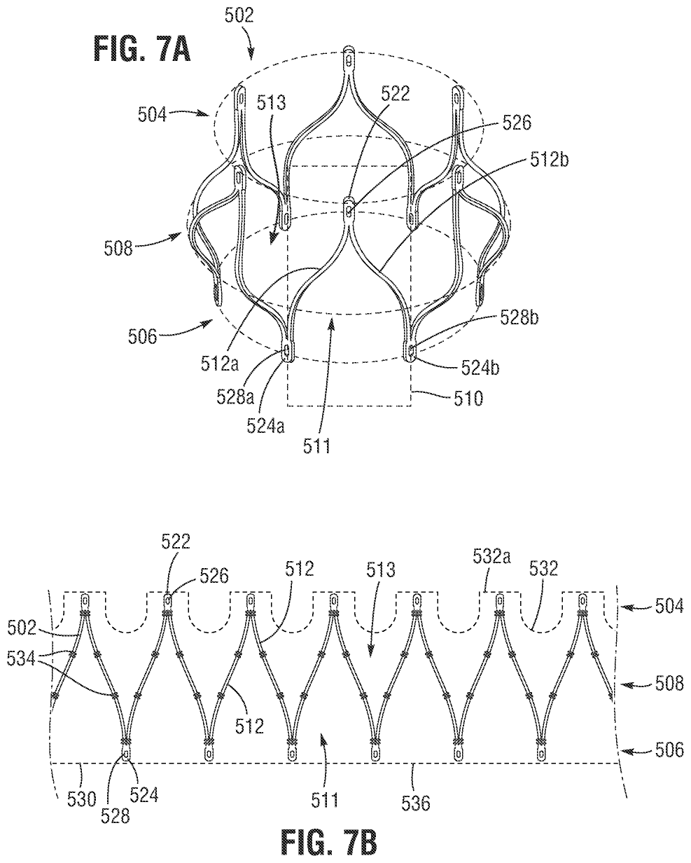

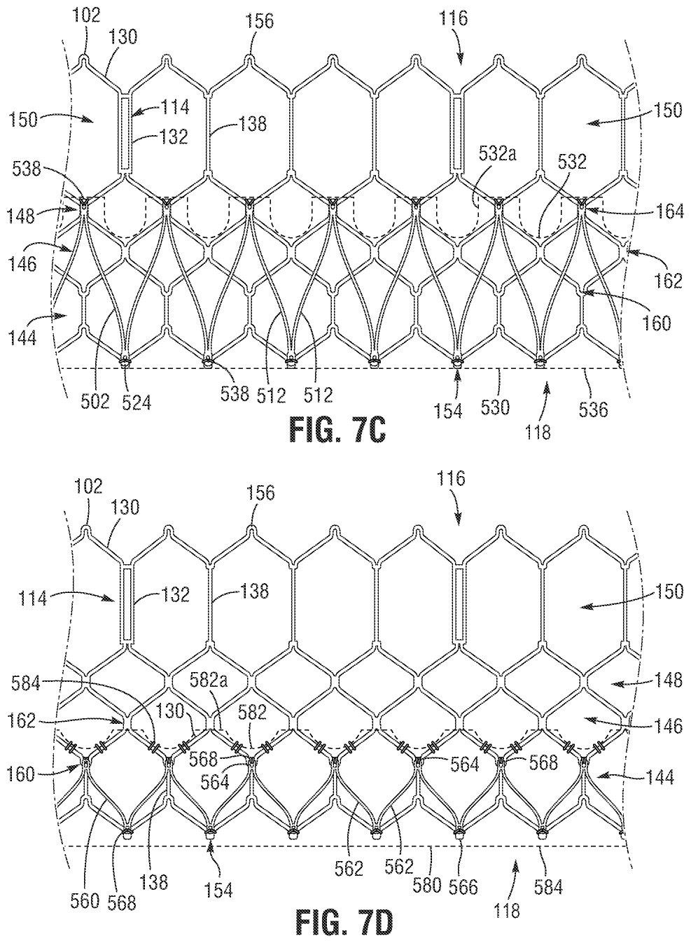

In some examples, a sealing frame can be formed by a network of struts that connect together to form open cells (e.g., open along the axial direction with respect to an inflow end or outflow end of the valve). FIG. 7A illustrates an example of such a sealing frame 502 with open cells in an expanded configuration. FIG. 7B shows an exemplary arrangement of and attachment between the sealing frame 502 and outer skirt 530. FIG. 7C shows an exemplary arrangement of the expanded sealing frame 502 with respect to the valve frame 102 and outer skirt 530.

In the illustrated example of FIG. 7A, the sealing frame 502 is formed by a plurality of elementary units 510 arrayed around a circumference of the valve frame and coupled together. Each elementary unit 510 can have a first apex 522 at one axial end and a pair of second apices 524a, 524b at an opposite axial end. The first apices 522 of the elementary units 510 can define a first axial end portion 504 that is coupled to the valve frame 102 (e.g., at a location remote from the inflow end 118), and the second apices 524 of the elementary units 510 can define a second axial end portion 506 that is coupled to the valve frame 102 (e.g., at or near the inflow end 118). In the illustrated example of FIG. 7A, each of the first apices 522 has a first eyelet 526, and each of the second apices 524a, 524b has a second eyelet 528a, 528b. One or more sutures (e.g., sutures 538 in FIG. 7C) can be passed through the eyelets 526, 528 in order to couple the respective apices 522, 524 to portions of the valve frame 102. Alternatively, instead of providing eyelets 526, 528, sutures can be wrapped around the respective apices 522, 524.

Each elementary unit 510 can have a first angled strut 512a and a second angled strut 512b. The first and second angled struts 512a, 512b are connected to each other via first apex 522 and form cell 511 that opens toward an inflow end of the valve frame 102. Each second apex 524 can be shared with adjacent elementary units 510 to form the sealing frame 502 as a continuous array of elementary units 510 that surrounds an outer circumference of the valve frame 102. The angled struts 512 of adjacent elementary units 510 that share a second apex 524 can form another cell 513 that opens toward an outflow end of the valve frame 102. In some examples, a middle portion of each angled strut 512 of the elementary units 510 can be considered an intermediate portion 508 that projects radially outward from the valve frame, as illustrated in FIG. 7A, when the sealing frame is in the expanded configuration. In the expanded configuration, the angled struts 512 may thus have a curved shape, such as C-shape, in side view.

In the expanded configuration of the sealing frame 502 shown in FIG. 7A, the middle portions of the angled struts 512 are spaced apart from each other along the circumferential direction. However, when the sealing frame 502 transitions to the compressed configuration, the apices 522, 524 move away from each other with respect to the axial direction of the valve frame 102 and the middle portions of the angled struts 512 move toward each other along the circumferential direction. The first apices 522 move toward each other along the circumferential direction to close, or at least reduce an opening of, cells 513, while the second apices 524 move toward each other along the circumferential direction to close, or at least reduce an opening of, cells 511. The final compressed configuration may be a state where first apices 522 are in contact with each other (or almost touching), where second apices 524 are in contact with each other (or almost touching), and/or where each angled strut 512 is substantially parallel to an axial direction of the valve frame 102. Alternatively or additionally, the final compressed configuration may be a state where the first axial end portion 504 and the second axial end portion 506 have an inner diameter that substantially matches an outer diameter of the valve frame 102 in its compressed configuration. With sealing frame 502 in its final compressed configuration, the radial projection of intermediate portion 508 can be eliminated, or at least reduced, due to the positioning of the angled struts 512 of each elementary unit 510. Thus, the cell configuration of the sealing frame 502 can allow it to adopt a low profile (e.g., minimal diameter) in the compressed configuration for transcatheter delivery to the implantation site, and to adopt a radially-projecting profile in the expanded configuration once delivered to the implantation site. The open-cell configuration of sealing frame 502 may allow it to adopt a lower profile (e.g., smaller diameter) in the compressed configuration than the closed-cell configurations of sealing frames illustrated in FIGS. 5A-6B.

In the illustrated example of FIG. 7A, each elementary unit 510 is symmetric with respect to an axially-extending centerline through first apex 522 (e.g., extending mid-way between second apices 524a, 524b). The geometry extending from each second apex 524 may also be an offset mirror image of the geometry extending from each first apex 522. However, in some examples, each elementary unit 510 may be asymmetrical with respect to one or more directions. For example, the shape of the angled struts 512a, 512b may be such that the protruding intermediate portion 508 is disposed closer to an inflow end 118 of the valve frame 102.

In some examples, the sealing frame 502 is constructed of a shape memory material, such as a nickel titanium alloy (e.g., Nitinol). The sealing frame 502 can be constructed such that its original pre-deformed shape has the intermediate portion 508 projecting radially outward (e.g., as compared to the radial locations of the first axial end portion 504, the second axial end portion 506, and/or a radially-outer circumferential surface of the valve frame 102). The sealing frame 502 can then transition to its compressed configuration, for example, by radially compressing the intermediate portion 508 and/or by displacing the first axial end portion 504 and the second axial portion 506 away from each other. By exposing the sealing frame 502 in the compressed configuration to a temperature in excess of its transition temperature and after release from any external restriction (e.g., a sheath of the delivery apparatus), the sealing frame 502 automatically reverts to is original pre-deformed shape, e.g., the profile for the expanded configuration illustrated in FIG. 7A. The transition temperature of the shape memory material may be tailored by appropriate selection of the material composition. In some examples, the shape memory alloy has a transition temperature (e.g., 30° C.) that is less than a normal body temperature of the patient (e.g., 37° C.).

The sealing frame 502 can be constructed by any number of fabrication techniques. In some examples, the sealing frame 502 can be constructed by laser cutting a shape memory material tube to form the angled struts 512, first apices 522, and second apices 524. Alternatively or additionally, in some examples, the sealing frame 502 can be constructed by laser welding of individual shape memory material wires together, for example, where a first wire forms the first angled strut 512a, and a second wire forms the second angled strut 512b. In either example, laser cutting or mechanical machining can be used to form the first eyelets 526 and the second eyelets 528. In some examples, after forming the interconnected strut structure, intermediate portion 508 of the sealing frame 502 can be modified such that it projects radially outward, as illustrated in FIG. 7A. In some examples, the intermediate portion 508 can be displaced radially outward from the first and second axial end portions 504, 506 (e.g., by using a preformed or inflatable mandrel) while the sealing frame 502 is held at a temperature in excess of the transition temperature. Alternatively or additionally, the first and second axial portions 504, 506 can be displaced radially inward from the intermediate portion 508 (e.g., by using a mandrel, rollers, and/or a lathe). The sealing frame 502 can then be cooled to a temperature below the transition temperature to set its current shape as the original pre-deformed shape. Alternatively or additionally, individual wires can be bent above their transition temperatures to have appropriate protruding portions that correspond to the desired intermediate portion 508. The wires can then be coupled together, for example, by laser welding, to form the interconnected strut structure for sealing frame 502. Deformation of the sealing frame 502 while below the transition temperature (e.g., in transitioning to the compressed configuration) can be effectively undone by heating to a temperature about the transition temperature, whereby the sealing frame 502 automatically reverts to its original pre-deformed shape. Further details regarding shape memory material fabrication techniques, which can be used to form sealing frame 502, can be found in U.S. Pat. Nos. 5,540,712 and 8,187,396, each of which is incorporated herein by reference.

In some examples, the outer skirt may be coupled to the sealing frame prior to attachment of the sealing frame to the valve frame. For example, one or more sutures may be used to attach the outer skirt to strut portions of the sealing frame. In some examples, the sutures can follow a perimeter of each elementary unit cell and wrap around the angled struts defining the elementary unit cell. Alternatively, the outer skirt can be attached to the angled struts via respective sutures close to or at the axial ends defined by the apices, without any direct coupling between the sealing frame and the portion of the outer skirt corresponding to the intermediate portion. For example, outer skirt 530 is attached to sealing frame 502 via a plurality of sutures 534 that wrap around angled struts 512 near the first apices 522 and the second apices 524, as shown in FIG. 7B. However, a central portion of the outer skirt 530 corresponding to intermediate portion 508 of the sealing frame 502 can remain uncoupled, for example, such that the outer skirt 530 can accommodate changes in shape of the sealing frame as it transitions between expanded and compressed configurations.

The outer skirt 530 can have a first edge portion 536 positioned closest to the inflow end 118 of the valve frame 102. In some examples, the first edge portion 536 can be separately coupled to the valve frame 102. For example, the first edge portion 536 can be wrapped around the inflow end 118 of the valve frame 102 to be attached via one or more sutures to a radially-inner circumferential surface of the valve frame 102, or to an inner skirt (e.g., inner skirt 108) on the radially-inner circumferential surface of the valve frame 102. In some examples, opposite to the first edge portion 536 along the axial direction, the outer skirt 530 can have a second edge portion 532 positioned closest to the outflow end 116 of the valve frame 102. In some examples, the second edge portion 532 is a patterned edge portion with portions 532a that project axially toward the outflow end 116 of the valve frame 102. For example, each axially-projecting portion 532a can be aligned with a respective one of the first apices 522, as shown in FIG. 7B. Alternatively, in some examples, the second edge portion of the outer skirt may follow a substantially straight edge (e.g., with the edge extending around the circumference of the valve frame 102 at a substantially constant distance with respect to the inflow end 118 or the outflow end 116).

In some examples, the sealing frame 502 can be constructed and attached to the valve frame 102 such that each elementary unit 510 corresponds to a circumferential row of rung junctions of the valve frame. For example, the first apices can correspond to rung junctions of the valve frame in a one-to-one correspondence, and the second apices can correspond to inflow apices of the valve frame in a one-to-one correspondence. As illustrated in FIG. 7C, each first apex 522 can thus be attached to a respective third rung junction 164 of the valve frame 102, and each second apex 524 can be attached to an inflow-end apex 154 of the valve frame 102. One or more sutures 538 can pass through eyelets 526 of the first apices 522 and wrap around the third rung junctions 164 to securely couple the first axial end portion 504 of the sealing frame 502 to the valve frame 102. One or more sutures 538 can pass through eyelets 528 of the second apices 524 and wrap around the inflow-end apices 154 to securely couple the second axial end portion 506 of the sealing frame 502 to the valve frame 102.

Since the first apex 522 is offset along the circumferential direction from the second apex 524, the sealing frame 502 is not restricted to attachment to rung junctions aligned with the inflow apices 154 of the valve frame 102. Thus, the first axial end portion 504 of the sealing frame 502 can be coupled to the third rung junction 164 (as shown in FIG. 7C) or the first rung junction 160 (e.g., as shown in FIG. 7D). In contrast, the sealing frames illustrated in FIGS. 5A-6B have first and second apices that are aligned and thus are restricted to rung junctions aligned with the inflow apices 154 (e.g., the second rung junction 162 and the fourth rung junction 166) for attachment.

In some examples, once the sealing frame 502 with outer skirt 530 coupled thereto is attached to the valve frame 102, the outer skirt 530 can then be separately coupled to the valve frame 102. For example, as described above, part of the outer skirt can be wrapped around the inflow end 118 of the valve frame 102 to be coupled at a radially-inner side of the valve frame 102. Alternatively or additionally, part of the outer skirt can be separately coupled at a radially-outer side of the valve frame, for example, by suturing the first edge portion to angled struts 130 extending from inflow apices 154 of the valve frame 102. It should be noted that the illustration in FIG. 7C shows the outer skirt 530 in dashed outline only to avoid obscuring the underlying sealing and valve frame structures. FIG. 7C thus does not show coupling of the outer skirt to the sealing frame 502 (e.g., via sutures 538) or to the valve frame 102.

In FIG. 7C, the outer skirt 530 is coupled to the sealing frame 502. Alternatively, in some examples, the outer skirt can be coupled to the valve frame 102 instead of the sealing frame 502, for example, as illustrated in FIG. 7D. In addition, FIG. 7D illustrates a variation of the sealing frame of FIG. 7C, in particular, to shorten an axial height of the sealing frame. The sealing frame 560 can be coupled to valve frame 102 in a manner similar to that described above with respect to FIG. 7C, for example, by using sutures 568 to attach first apices 564 to first rung junction 160 and to attach second apices 566 to inflow apices 154 of the valve frame 102. Each elementary unit of the sealing frame 560 may thus correspond to a respective cell 144 of the first row of the valve frame 102. In a side view of the sealing frame 560 along a radial direction, angled struts 562 of each elementary unit may appear contained within the bounds of the cell 144 of the valve frame 102.

Once the sealing frame 502 is secured to the valve frame 102, an outer skirt 580 can then be draped over the sealing frame 502. Similar to the outer skirt 530 illustrated in FIGS. 7B-7C, outer skirt 580 can have a first edge portion positioned closest to the inflow edge 118 of the valve frame 102 and an opposite second edge portion 582 positioned closest to the outflow edge 116 of the valve frame 102. The first edge portion of the outer skirt 580 can be wrapped around the inflow end 118 of the valve frame 102 for attachment on a radially-inner side of the valve frame 102 (e.g., to inner skirt 108). As shown in FIG. 7D, in some examples, the second edge portion 582 can be patterned with portions 582a that project axially toward the outflow end 116 of the valve frame 102. However, in contrast to the configuration of outer skirt 530 in FIGS. 7B-7C, the outer skirt 580 extends along the axial direction of the valve frame 102 beyond the first apices 564 of the sealing frame 502. In addition, instead of being aligned with the first apices 564, axially-projecting portions 582a can be offset from the first apices 564 along a circumferential direction of the valve frame 102. The projection portions 582a can thus extend to and be aligned with respective second rung junctions 162, where one or more sutures 584 can be used to attach the projecting portions 582a to the valve frame 102 (e.g., angled struts 130 extending from second rung junction 162). It should be noted that the illustration in FIG. 7D shows the outer skirt 580 in dashed outline only to avoid obscuring the underlying sealing and valve frame structures. Some sutures 584 that attach the second end portion 582 of the outer skirt 580 to the valve frame 102 are also shown, although additional sutures and/or different suture configurations are also possible.

In the examples illustrated in FIGS. 7C-7D, the second apices of the sealing frame (e.g., apices at the inflow end) have a one-to-one correspondence with the inflow apices 154 of the valve frame 102, and the first apices of the sealing frame (e.g., apices at the outflow end) have a one-to-one correspondence with junctions (e.g., first rung junction 160 or third rung junction 164). Alternatively, in some examples, the correspondence between the apices of the sealing frame and the apices/junctions of the valve frame 102 can be other than one-to-one. For example, in a manner similar to that illustrated in FIG. 5G, the second apices of the sealing frame having the shape illustrated in FIG. 7A can be attached to every other one of the inflow apices 154 and the first apices of this sealing frame can be attached to every other one of the rung junctions in a particular circumferential row of the valve frame 102.

Although specific examples are discussed above, other examples are also possible in one or more implementations. FIGS. 7B-7C describe the outer skirt being directly attached to the sealing frame prior to attachment of the sealing frame to the valve frame. However, it is also possible for the sealing frame to be attached to the valve frame first, and then attach the outer skirt directly to the sealing frame. Alternatively or additionally, the outer skirt in any of FIGS. 7B-7C can be modified to attach directly to the valve frame, for example, in a manner similar to that described above with respect to FIG. 5E or 7D.

It should also be noted that the illustrations in FIGS. 7B-7D show the sealing frame, outer skirt, and valve frame in a flat planar layout for convenience only. In particular implementations, the sealing frame will have the three-dimensional profile when in the expanded configuration (e.g., the profile illustrated in FIG. 7A), and the valve frame 102 will have the annular configuration illustrated in FIGS. 3B and 4C. The coupling of the outer skirt to the sealing frame may occur with the sealing frame having such a three-dimensional profile, and the coupling of the sealing frame to the annular valve frame may occur with the sealing frame having such a three-dimensional profile.

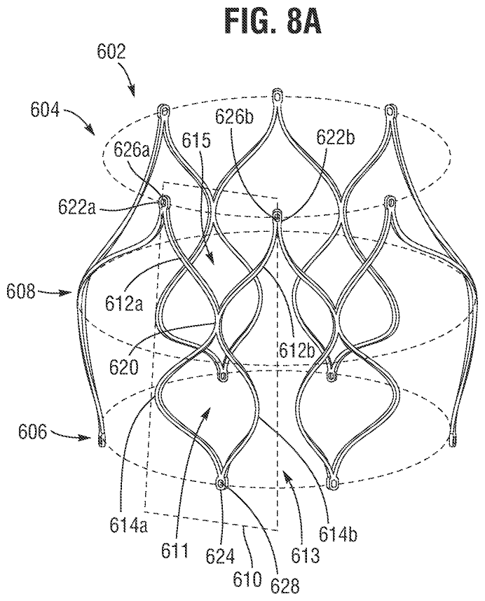

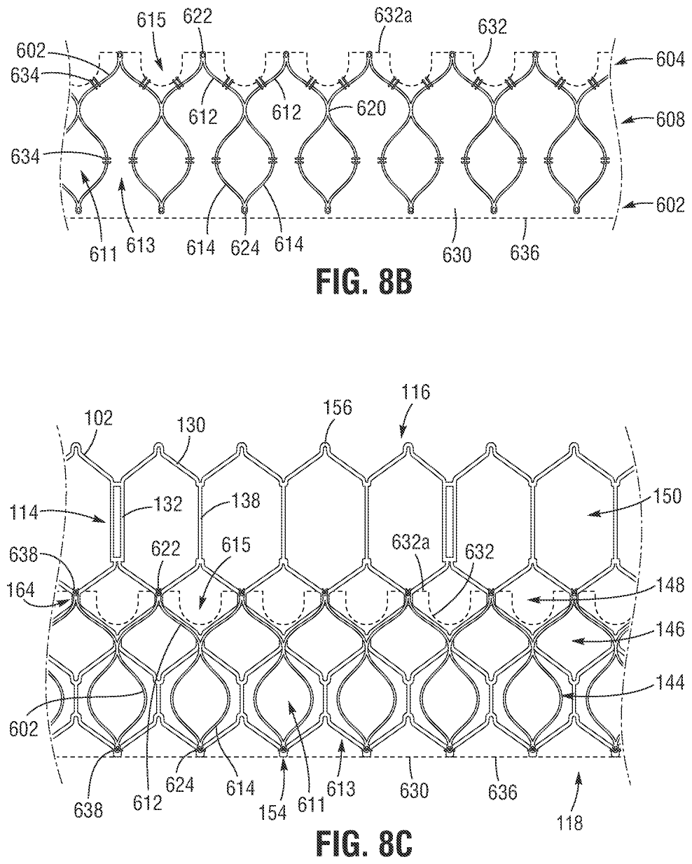

In some examples, sealing frames with open cells may allow for a lower profile in the compressed configuration as compared to sealing frames with closed cells, and sealing frames with closed cells may provide a more rigid structure in the expanded configuration as compared to sealing frames with open cells. In some examples, sealing frames with open calls may allow for attachment points to the valve frame that are different than those offered by sealing frames with closed cells. Thus, in some examples, a sealing frame can be formed by a network of struts that connect together to form both open cells and closed cells, thereby taking advantage of features of each cell configuration. FIG. 8A illustrates an example of such a sealing frame 602 with open and closed cells in an expanded configuration. FIG. 8B shows an exemplary arrangement of and attachment between the sealing frame 602 and outer skirt 630. FIG. 8C shows an exemplary arrangement of the expanded sealing frame 602 with respect to the valve frame 102 and outer skirt 630.

In the illustrated example of FIG. 8A, the sealing frame 602 is formed by a plurality of elementary units 610 arrayed around a circumference of the valve frame and coupled together. Each elementary unit 610 can have a pair of first apices 622a, 622b at one axial end and a second apex 624 at an opposite axial end. The first apices 622a, 622b of the elementary units 610 can define a first axial end portion 604 that is coupled to the valve frame 102 (e.g., at a location remote from the inflow end 118), and the second apices 624 of the elementary units 610 can define a second axial end portion 606 that is coupled to the valve frame 102 (e.g., at or near the inflow end 118). In the illustrated example of FIG. 8A, each of the first apices 622a, 622b has a first eyelet 626a, 626b, and each of the second apices 624 has a second eyelet 628. One or more sutures (e.g., sutures 638 in FIG. 8C) can be passed through the eyelets 626, 628 in order to couple the respective apices 622, 624 to portions of the valve frame 102. Alternatively, instead of providing eyelets 626, 628, sutures can be wrapped around the respective apices 622, 624.

Each elementary unit 610 can have a first angled strut 612a and a second angled strut 612b. The first angled strut 612a extends from first apex 622a to coupling portion 620, and the second angled strut 612b extends from first apex 622b to coupling portion 620. The first and second angled struts 612a, 612b are thus connected to each other via coupling portion 620 and form cell 615 that opens toward an inflow end of the valve frame 102. Each first apex 622 can be shared with adjacent elementary units 610 to form the sealing frame 602 as a continuous array of elementary units 610 that surrounds an outer circumference of the valve frame 102. Each elementary unit 610 can also have a first curved strut 614a and a second curved strut 614b. The first curved strut 614a extends from second apex 624 to coupling portion 620, and the second curved strut 614b extends from second apex 624 to coupling portion 620. The first and second curved struts 614a, 614b are thus connected together at opposite ends via coupling portion 620 and second apex 624 and form closed cell 611. The angled struts 612 of adjacent elementary units 610 that share a first apex 622 can form another cell 613 that opens toward an outflow end of the valve frame 102. Alternatively, in some examples, cell 613 formed by angled struts 612 of adjacent elementary units 610 can be closed off to the outflow end of the valve frame by coupling together adjacent portions of the curved struts 614 (e.g., by coupling a mid-portion of the first curved strut 614a of one elementary unit 610 to a mid-portion of the second curved strut 614b of an adjacent elementary unit 610).

In some examples, a middle portion of each curved strut 614 of the elementary units 610 can be considered an intermediate portion that projects radially outward from the valve frame when the sealing frame 602 is in the expanded configuration. In the expanded configuration, the angled struts 614 may thus have a curved shape, such as C-shape, in side view. Alternatively, in some examples, the coupling portions 620 of the elementary units 610 can be considered the intermediate portion that projects radially outward from the valve frame when the sealing frame 602 is in the expanded configuration. Alternatively, in some examples, the intermediate portion is at a location along the axial direction between a middle portion of each curved strut 614 and coupling portion 620, as illustrated in FIG. 8A. In the illustrated example of FIG. 8A, each elementary unit 610 is symmetric with respect to an axially-extending centerline between the second apex 624 and the coupling portion 620. However, in some examples, each elementary unit 510 may be asymmetrical with respect to one or more directions.

In the expanded configuration of the sealing frame 602 shown in FIG. 8A, the middle portions of the angled struts 612 are spaced apart from each other along the circumferential direction, and the middle portions of the curved struts 614 are spaced apart from each other along the circumferential direction. However, when the sealing frame 602 transitions to the compressed configuration, the apices 622, 624 move away from each other with respect to the axial direction of the valve frame 102. The middle portions of the angled struts 612 move toward each other along the circumferential direction, and the middle portions of curved struts 614 move toward each other along the circumferential direction. The first apices 622 move toward each other along the circumferential direction to close, or at least reduce an opening of, cells 615, The second apices 624 move toward each other along the circumferential direction to close, or at least reduce an opening of, cells 613. The movement of angled struts 612 toward each other may also serve to eliminate closed cell 611, or at least reduce a maximum dimension in the circumferential direction of cell 611. The final compressed configuration may be a state where apices 622 are in contact with each other (or almost touching), where apices 624 are in contact with each other (or almost touching), where each curved strut and/or where each angled strut 512 is substantially parallel to an axial direction of the valve frame 102. Alternatively or additionally, the final compressed configuration may be a state where the first axial end portion 604 and the second axial end portion 606 have an inner diameter that substantially matches an outer diameter of the valve frame 102 in its compressed configuration. With the sealing frame 602 in its final compressed configuration, the radial projection of intermediate portion 608 can be eliminated, or at least reduced, due to the positioning of the angled struts 612 and/or curved struts 614 of each elementary unit 610. Thus, the cell configuration of the sealing frame 602 can allow it to adopt a low profile (e.g., minimal diameter) in the compressed configuration for transcatheter delivery to the implantation site, and to adopt a radially-projecting profile in the expanded configuration once delivered to the implantation site.

In some examples, the sealing frame 602 is constructed of a shape memory material, such as a nickel titanium alloy (e.g., Nitinol). The sealing frame 602 can be constructed such that its original pre-deformed shape has the intermediate portion 608 projecting radially outward (e.g., as compared to the radial locations of the first axial end portion 604, the second axial end portion 606, and/or a radially-outer circumferential surface of the valve frame 102). The sealing frame 602 can then transition to its compressed configuration, for example, by radially compressing the intermediate portion 608 and/or by displacing the first axial end portion 604 and the second axial end portion 606 away from each other. By exposing the sealing frame 602 in the compressed configuration to a temperature in excess of its transition temperature and after release from any external restriction (e.g., a sheath of the delivery apparatus), the sealing frame 602 automatically reverts to is original pre-deformed shape, e.g., the profile for the expanded configuration illustrated in FIG. 8A. The transition temperature of the shape memory material may be tailored by appropriate selection of the material composition. In some examples, the shape memory alloy has a transition temperature (e.g., 30° C.) that is less than a normal body temperature of the patient (e.g., 37° C.).

The sealing frame 602 can be constructed by any number of fabrication techniques. For example, in some examples, the sealing frame 602 is constructed by laser cutting a shape memory material tube to form the angled struts 612, curved struts 614, coupling portions 620, first apices 622, and second apices 624. Alternatively or additionally, in some examples, the sealing frame 602 can be constructed by laser welding of individual shape memory material wires together, for example, where a first wire forms the first angled strut 612a and first curved strut 614a, and a second wire forms the second angled strut 612b and the second curved strut 614b. The first and second wires can then be joined at ends to form apices 622, 624 and at mid-portions to form coupling portions 620. Alternatively or additionally, in some examples, a first wire can form the first angled strut 612a, a second wire can form the second angled strut 612b, and a third wire can be bent and coupled to itself to form the first curved strut 614a and second curved strut 614b of the closed cell 611. Alternatively or additionally, in some examples, a single wire can be bent to form the various struts 612, 614 of the elementary unit 610 and the single wire can be joined to itself to form coupling portion 620, second apex 624, and/or first apices. In any of the above noted examples, laser cutting or mechanical machining can be used to form the first eyelets 526 and the second eyelets 528.

In some examples, after forming the interconnected strut structure, intermediate portion 608 of the sealing frame 602 can be modified such that it projects radially outward, as illustrated in FIG. 8A. In some examples, the intermediate portion 608 can be displaced radially outward from the first and second axial end portions 604, 606 (e.g., by using a preformed or inflatable mandrel) while the sealing frame 602 is held at a temperature in excess of the transition temperature. Alternatively or additionally, the first and second axial end portions 604, 606 can be displaced radially inward from the intermediate portion 608 (e.g., by using a mandrel, rollers, and/or a lathe). The sealing frame 602 can then be cooled to a temperature below the transition temperature to set its current shape as the original pre-deformed shape. Alternatively or additionally, individual wires can be bent above their transition temperatures to have appropriate protruding portions that correspond to the desired intermediate portion 608. The wires can then be coupled together, for example, by laser welding, to form the interconnected strut structure for sealing frame 602. Deformation of the sealing frame 602 while below the transition temperature (e.g., in transitioning to the compressed configuration) can be effectively undone by heating to a temperature about the transition temperature, whereby the sealing frame 602 automatically reverts to its original pre-deformed shape. Further details regarding shape memory material fabrication techniques, which can be used to form sealing frame 602, can be found in U.S. Pat. Nos. 5,540,712 and 8,187,396, each of which is incorporated herein by reference.

In some examples, the outer skirt may be coupled to the sealing frame prior to attachment of the sealing frame to the valve frame. For example, one or more sutures may be used to attach the outer skirt to strut portions of the sealing frame. In some examples, the sutures can follow a perimeter of each elementary unit cell and wrap around the angled struts defining the elementary unit cell. Alternatively, the outer skirt can be attached to the angled struts via respective sutures close to or at the axial ends defined by the apices, without any direct coupling between the sealing frame and the portion of the outer skirt corresponding to the intermediate portion. For example, outer skirt 630 is attached to sealing frame 602 via a plurality of sutures 634 that wrap around angled struts 612 near the first apices 622 and around mid-portions of the curved struts 614. However, a central portion of the outer skirt 630 corresponding to intermediate portion 608 of the sealing frame 602 can remain uncoupled, for example, such that the outer skirt 630 can accommodate changes in shape of the sealing frame as it transitions between expanded and compressed configurations.

The outer skirt 630 can have a first edge portion 636 positioned closest to the inflow end 118 of the valve frame 102. In some examples, the first edge portion 636 can be separately coupled to the valve frame 102. For example, the first edge portion 636 can be wrapped around the inflow end 118 of the valve frame 102 to be attached via one or more sutures to a radially-inner circumferential surface of the valve frame 102, or to an inner skirt (e.g., inner skirt 108) on the radially-inner circumferential surface of the valve frame 102. In some examples, opposite to the first edge portion 636 along the axial direction, the outer skirt 630 can have a second edge portion 632 positioned closest to the outflow end 116 of the valve frame 102. In some examples, the second edge portion 632 is a patterned edge portion with portions 632a that project axially toward the outflow end 116 of the valve frame 102. For example, each axially-projecting portion 632a can be aligned with a respective one of the first apices 622, as shown in FIG. 8B. Alternatively, in some examples, the second edge portion of the outer skirt may follow a substantially straight edge (e.g., with the edge extending around the circumference of the valve frame 102 at a substantially constant distance with respect to the inflow end 118 or the outflow end 116).

In some examples, the sealing frame 602 can be constructed and attached to the valve frame 102 such that each elementary unit 610 corresponds to a circumferential row of rung junctions of the valve frame. For example, the first apices can correspond to rung junctions of the valve frame in a one-to-one correspondence, and the second apices can correspond to inflow apices of the valve frame in a one-to-one correspondence. As illustrated in FIG. 8C, each first apex 622 can thus be attached to a respective third rung junction 164 of the valve frame 102, and each second apex 624 can be attached to an inflow-end apex 154 of the valve frame 102. One or more sutures 638 can pass through eyelets 626 of the first apices 622 and wrap around the third rung junctions 164 to securely couple the first axial end portion 604 of the sealing frame 602 to the valve frame 102. One or more sutures 638 can pass through eyelets 628 of the second apices 624 and wrap around the inflow-end apices 154 to securely couple the second axial end portion 606 of the sealing frame 602 to the valve frame 102.

Since the first apex 622 is offset along the circumferential direction from the second apex 624, the sealing frame 602 is not restricted to attachment to rung junctions aligned with the inflow apices 154 of the valve frame 102. Thus, the first axial end portion 604 of the sealing frame 602 can be coupled to the third rung junction 164 (as shown in FIG. 8C) or the first rung junction 160 (e.g., similar to the arrangement of sealing frame 560 in FIG. 7D). In contrast, the sealing frames illustrated in FIGS. 5A-6B have first and second apices that are aligned and thus are restricted to rung junctions aligned with the inflow apices 154 (e.g., the second rung junction 162 and the fourth rung junction 166) for attachment.

In some examples, once the sealing frame 602 with outer skirt 630 coupled thereto is attached to the valve frame 102, the outer skirt 630 can then be separately coupled to the valve frame 102. For example, as described above, part of the outer skirt can be wrapped around the inflow end 118 of the valve frame 102 to be coupled at a radially-inner side of the valve frame 102. Alternatively or additionally, part of the outer skirt can be separately coupled at a radially-outer side of the valve frame, for example, by suturing the first edge portion to angled struts 130 extending from inflow apices 154 of the valve frame 102. It should be noted that the illustration in FIG. 8C shows the outer skirt 630 in dashed outline only to avoid obscuring the underlying sealing and valve frame structures. FIG. 8C thus does not show coupling of the outer skirt to the sealing frame 602 (e.g., via sutures 638) or to the valve frame 102.

In FIG. 8C, the outer skirt 630 is coupled to the sealing frame 602. Alternatively, in some examples, the outer skirt can be coupled to the valve frame 102 instead of the sealing frame 602, for example, in a manner similar to that described above with respect to FIGS. 5E and 7D. In addition, the second apices 624 of the sealing frame 602 have a one-to-one correspondence with the inflow apices 154 of the valve frame 102, and the first apices 622 of the sealing frame 602 have a one-to-one correspondence with junctions (e.g., first rung junction 160 or third rung junction 164). Alternatively, in some examples, the correspondence between the apices of the sealing frame and the apices/junctions of the valve frame 102 can be other than one-to-one. For example, in a manner similar to that illustrated in FIG. 5G, the second apices of the sealing frame having the shape illustrated in FIG. 8A can be attached to every other one of the inflow apices 154, and the first apices of this sealing frame can be attached to every other one of the rung junctions in a particular circumferential row of the valve frame 102.

Although specific examples are discussed above, other examples are also possible in one or more implementations. FIGS. 8B-8C describe the outer skirt being directly attached to the sealing frame prior to attachment of the sealing frame to the valve frame. However, it is also possible for the sealing frame to be attached to the valve frame first, and then attach the outer skirt directly to the sealing frame. Alternatively or additionally, the outer skirt in any of FIGS. 8B-8C can be modified to attach directly to the valve frame, for example, in a manner similar to that described above with respect to FIG. 5E or 7D.

It should also be noted that the illustrations in FIGS. 8B-8C show the sealing frame 602, outer skirt 630, and valve frame 102 in a flat planar layout for convenience only. In actual implementations, the sealing frame will have the three-dimensional profile when in the expanded configuration (e.g., the profile illustrated in FIG. 8A), and the valve frame 102 will have the annular configuration illustrated in FIGS. 3B and 4C. The coupling of the outer skirt to the sealing frame may occur with the sealing frame having such a three-dimensional profile, and the coupling of the sealing frame to the annular valve frame may occur with the sealing frame having such a three-dimensional profile.

FIG. 9 shows an exemplary delivery apparatus 900 that can be used to deliver and implant prosthetic heart valve 800. Prosthetic heart valve 800 may be any of the prosthetic heart valves explicitly discussed above with respect to FIGS. 3A-8C, or any other prosthetic heart valve that includes a sealing frame. Delivery apparatus 900 includes a handle 902 that can be disposed external to a patient and used to articulate a distal end portion 906 of an elongated shaft within the patient. The prosthetic heart valve 800 can be disposed on the distal end portion 906 in its compressed state or configuration. That is, the valve frame as well as the associated sealing frame coupled to the valve frame can be in their respective compressed configurations. For example, the prosthetic valve 800 can be crimped on an inflatable balloon 904 or another type of expansion member that can be used to radially expand the prosthetic valve 800. The distal end portion 906, including prosthetic valve 800, can be advanced through the vasculature to a selected implantation site (e.g., within a native mitral valve and/or within a previously implanted host valve). Although not specifically illustrated in FIG. 9, it should be appreciated that the distal end portion 906 of the delivery apparatus 900 can be advanced over a guidewire, and that the delivery apparatus 900 can include an innermost shaft that defines a lumen for the guidewire, as is known in the art. The prosthetic valve 800 can then be deployed at the implantation site, such as by inflating the balloon 904. Further details of delivery apparatuses that can be used to deliver and implant plastically-expandable prosthetic heart valves, such as the prosthetic valve 800, are disclosed in U.S. Patent Application Publication Nos. 2017/0065415, 2016/0158497, and 2013/0030519, which are incorporated herein by reference.

If the prosthetic valve 800 being implanted is a self-expandable prosthetic valve, the prosthetic valve can be retained in a compressed configuration within a delivery capsule or sheath of the delivery apparatus 900 when inserted into and advanced through the patient's vasculature to the desired implantation site. In such configurations, the prosthetic valve 800 can be disposed within the delivery apparatus without provision of a balloon 904 or other expansion device. Once positioned at the desired implantation site, the prosthetic valve can be deployed from the delivery capsule, which allows the valve frame and the sealing frame of the prosthetic valve to each self-expand to their expanded, functional sizes within the native valve or a previously-implanted host valve. Further details of delivery apparatuses that can be used to deliver and implant self-expandable prosthetic valves are disclosed in U.S. Pat. Nos. 8,652,202 and 9,867,700, which are incorporated by reference herein.

When prosthetic valve 800 is implanted at the mitral location, a valve dock (e.g., docking station 152) can be used. For example, the valve dock can first be advanced and delivered to the native mitral valve annulus, and then set at a desired position, prior to implantation of the prosthetic heart valve 800. In some examples, the valve dock can be flexible and/or made of a shape memory material, so that the coils of the valve dock can be straightened for delivery via a transcatheter approach as well. In some examples, the coil is made of another biocompatible material, such as stainless steel. Some of the same catheters and other delivery tools can be used for both delivery of the valve dock 152 and the prosthetic valve 800, without having to perform separate preparatory steps, simplifying the implantation procedure for the end user. Further details of docking stations and implantation thereof, which may be employed with prosthetic valve 800 or any other exemplary valve, are disclosed in U.S. Pat. No. 10,463,479 and International Application No. PCT/US2020/036577, both of which are incorporated by reference herein.

### Additional Examples

Example 1. A prosthetic heart valve comprises a valve frame that is radially collapsible and expandable between a first compressed configuration and a first expanded configuration, the valve frame having an inflow end and an outflow end separated from the inflow end along an axial direction of the valve frame; a valvular structure coupled to the valve frame and comprising a plurality of leaflets within the valve frame; a sealing frame surrounding a radially-outer surface portion of the valve frame, the sealing frame being collapsible and expandable between a second compressed configuration corresponding to the first compressed configuration of the valve frame and a second expanded configuration corresponding to the second expanded configuration of the valve frame, the sealing frame having a first axial end coupled to the valve frame at the inflow end, a second axial end coupled to the valve frame at a location between the inflow and outflow ends along the axial direction, and an intermediate portion between the first and second axial ends along the axial direction; and an outer skirt surrounding the sealing frame, wherein, with the valve frame and the sealing frame in the first and second expanded configurations, respectively, the intermediate portion projects radially outward from the valve frame, thereby displacing at least a portion of the outer skirt radially outward.
Example 2. The prosthetic heart valve of any example herein, particularly example 1, wherein, in transitioning between the second compressed configuration and the second expanded configuration within an anatomy of a patient, the intermediate portion of the sealing frame is constructed to urge the outer skirt into contact with surrounding native tissue.
Example 3. The prosthetic heart valve of any example herein, particularly any one of examples 1-2, wherein, with the valve frame and the sealing frame in the first and second compressed configurations, respectively, the first axial end, the second axial end, and the intermediate portion of the sealing frame are substantially adjacent to the radially-outer surface portion of the valve frame.
Example 4. The prosthetic heart valve of any example herein, particularly any one of examples 1-3, wherein, with the valve frame and the sealing frame in the first and second compressed configurations, respectively, the first axial end, the second axial end, and the intermediate portion of the sealing frame are substantially aligned along a direction substantially parallel to the axial direction.
Example 5. The prosthetic heart valve of any example herein, particularly any one of examples 1-4, wherein the sealing frame is formed of a shape memory material.
Example 6. The prosthetic heart valve of any example herein, particularly example 5, wherein the shape memory material comprises a nickel titanium alloy.
Example 7. The prosthetic heart valve of any example herein, particularly any one of examples 1-6, wherein the sealing frame in the second expanded configuration has a first height along the axial direction between the first and second axial ends, and the sealing frame in the second compressed configuration has a second height along the axial direction between the first and second axial ends that is greater than the first height.
Example 8. The prosthetic heart valve of any example herein, particularly example 7, wherein the second height is at least 1.2 times the first height.
Example 9. The prosthetic heart valve of any example herein, particularly any one of examples 7-8, wherein the second height is about 1.23-1.3 times the first height.
Example 10. The prosthetic heart valve of any example herein, particularly any one of examples 1-9, wherein, with the valve frame and the sealing frame in the first and second expanded configurations, respectively, the intermediate portion projects outward from the radially-outer surface portion of the valve frame by an amount along a radial direction of the valve frame that is at least 5% of a diameter of the valve frame.
Example 11. The prosthetic heart valve of any example herein, particularly example 10, wherein the amount of projection along the radial direction is 6-14% of the diameter of the valve frame.
Example 12. The prosthetic heart valve of any example herein, particularly any one of examples 1-11, wherein the intermediate portion projects outward from the radially-outer surface portion of the valve frame by amount 2-4 mm along the radial direction.
Example 13. The prosthetic heart valve of any example herein, particularly any one of examples 1-12, wherein the sealing frame comprises a continuous unitary structure that spans an entire outer circumference of the radially-outer surface portion of the valve frame.
Example 14. The prosthetic heart valve of any example herein, particularly any one of examples 1-13, wherein an inner diameter of the sealing frame at the first axial end is substantially the same as an outer diameter of the valve frame at the inflow end, and an inner diameter of the sealing frame at the second axial end is substantially the same as an outer diameter of the radially-outer surface portion of the valve frame.
Example 15. The prosthetic heart valve of any example herein, particularly example 14, wherein the inner diameter of the sealing frame at the first axial end is substantially the same as the inner diameter of the sealing frame at the second axial end.
Example 16. The prosthetic heart valve of any example herein, particularly any one of examples 1-15, wherein the valve frame comprises a plurality of first struts connected together at respective junctions to form an open-cell lattice structure, each cell being open along the radial direction of the valve frame, junctions at the inflow end forming first apices, and junctions at the outflow end forming second apices.
Example 17. The prosthetic heart valve of any example herein, particularly example 16, wherein the first apices are offset from the second apices with respect to a circumferential direction of the valve frame.
Example 18. The prosthetic heart valve of any example herein, particularly any one of examples 16-17, wherein the first axial end of the sealing frame comprises a plurality of first coupling apices, the second axial end of the sealing frame comprises a plurality of second coupling apices, and a plurality of strut portions interconnecting the first and second coupling apices.
Example 19. The prosthetic heart valve of any example herein, particularly example 18, wherein each of the first and second coupling apices has an eyelet.
Example 20. The prosthetic heart valve of any example herein, particularly example 19, wherein each of the first and second coupling apices is coupled to a respective portion of the valve frame via one or more sutures through the respective eyelet.
Example 21. The prosthetic heart valve of any example herein, particularly any one of examples 18-20, wherein at least some of the plurality of strut portions form respective cells that are closed along the axial direction.
Example 22. The prosthetic heart valve of any example herein, particularly any one of examples 18-21, wherein the intermediate portion of the sealing frame comprises longitudinally-extending coupling portions between adjacent strut portions.
Example 23. The prosthetic heart valve of any example herein, particularly any one of examples 18-22, wherein each first coupling apex is coupled to a respective one of the first apices of the valve frame, each second coupling apex is coupled to a respective one of the junctions of the first struts of the valve frame, and the first coupling apices are aligned with the second coupling apices, respectively, with respect to a circumferential direction of the valve frame.
Example 24. The prosthetic heart valve of any example herein, particularly any one of examples 16-23, wherein the sealing frame extends along the axial direction over at least part of a circumferential row of cells of the lattice structure of the valve frame.
Example 25. The prosthetic heart valve of any example herein, particularly any one of examples 16-24, wherein the sealing frame extends along the axial direction over a single circumferential rows of cells of the lattice structure of the valve frame.
Example 26. The prosthetic heart valve of any example herein, particularly any one of examples 16-25, wherein the sealing frame extends along the axial direction over at least two circumferential rows of cells of the lattice structure of the valve frame.
Example 27. The prosthetic heart valve of any example herein, particularly any one of examples 16-26, wherein the sealing frame extends along the axial direction over three circumferential rows of cells of the lattice structure of the valve frame.
Example 28. The prosthetic heart valve of any example herein, particularly any one of examples 18-27, wherein at least some of the plurality of strut portions form respective cells that are open along the axial direction.
Example 29. The prosthetic heart valve of any example herein, particularly example 28, wherein the first coupling apices are offset from the second coupling apices with respect to a circumferential direction of the valve frame.
Example 30. The prosthetic heart valve of any example herein, particularly any one of examples 16-17, wherein the sealing frame comprises elementary units arrayed around a circumference of the sealing frame, each elementary unit comprising first and second angled strut portions extending from a first apex at the first axial end and third and fourth angled strut portions extending from a second apex at the second axial end, the first apex being aligned with the second apex along the axial direction, the first angled strut portion joining with the third angled strut portion via a first coupling portion, the second angled strut portion joining with the fourth angled strut portion via a second coupling portion, adjacent elementary units in the array being joined together at adjacent coupling portions.
Example 31. The prosthetic heart valve of any example herein, particularly any one of examples 16-17, wherein the sealing frame comprises elementary units arrayed around a circumference of the sealing frame, each elementary unit comprising first and second angled strut portions extending from a first apex at the first axial end and third and fourth angled strut portions extending from a second apex at the second axial end, the first apex being aligned with the second apex along the axial direction, the first angled strut portion joining with the third angled strut portion via a first longitudinally-extending strut, the second angled strut portion joining with the fourth angled strut portion via a second longitudinally-extending strut, adjacent elementary units in the array being joined together at adjacent longitudinally-extending struts.
Example 32. The prosthetic heart valve of any example herein, particularly any one of examples 16-17, wherein the sealing frame comprises elementary units arrayed around a circumference of the sealing frame, each elementary unit comprising first and second angled struts extending from a first apex at the first axial end to respective second apices at the second axial end, adjacent elementary units in the array being joined together at adjacent second apices.
Example 33. The prosthetic heart valve of any example herein, particularly any one of examples 16-17, wherein the sealing frame comprises elementary units arrayed around a circumference of the sealing frame, each elementary unit comprising first and second curved struts extending from a first apex at the first axial end to a coupling portion and third and fourth angled strut portions extending from the coupling portion to respective second apices at the second axial end, adjacent elementary units in the array being joined together at adjacent second apices.
Example 34. The prosthetic heart valve of any example herein, particularly any one of examples 1-33, wherein the outer skirt extends along the axial direction from at least the inflow end of the valve frame to at least the second axial end of the sealing frame.
Example 35. The prosthetic heart valve of any example herein, particularly any one of examples 1-34, wherein the outer skirt is directly coupled to the sealing frame.
Example 36. The prosthetic heart valve of any example herein, particularly example 35, wherein one or more second sutures directly couple the outer skirt to corresponding portions of the sealing frame.
Example 37. The prosthetic heart valve of any example herein, particularly any one of examples 1-36, wherein at least the intermediate portion of the sealing frame is coupled to a facing portion of the outer skirt.
Example 38. The prosthetic heart valve of any example herein, particularly any one of examples 1-34, wherein the outer skirt is coupled to the valve frame without otherwise being coupled to the sealing frame.
Example 39. The prosthetic heart valve of any example herein, particularly example 38, wherein one or more third sutures directly couple the outer skirt to corresponding portions of the valve frame.
Example 40. The prosthetic heart valve of any example herein, particularly any one of examples 1-39, wherein the outer skirt extends along the axial direction from the inflow end of the valve frame farther than the sealing frame.
Example 41. The prosthetic heart valve of any example herein, particularly any one of examples 1-40, wherein an edge of the outer skirt opposite the inflow end of the valve frame has an undulating pattern with a plurality of axially projecting portions, and each projecting portion is aligned with a corresponding strut junction of the valve frame.
Example 42. The prosthetic heart valve of any example herein, particularly example 41, wherein apices of the sealing frame at the second axial end are aligned with the projecting portions of the undulating pattern, respectively, with respect to a circumferential direction of the valve frame.
Example 43. The prosthetic heart valve of any example herein, particularly example 41, wherein apices of the sealing frame at the second axial end are offset from the projecting portions of the undulating pattern, respectively, with respect to a circumferential direction of the valve frame.
Example 44. A prosthetic heart valve comprises a valve frame that is radially collapsible and expandable between a compressed configuration and an expanded configuration, the valve frame having an inflow end and an outflow end separated from the inflow end along an axial direction of the valve frame; a valvular structure coupled to the valve frame and comprising a plurality of leaflets within the valve frame; an outer skirt; and means for displacing at least a portion of the outer skirt radially outward from the valve frame.
Example 45. The prosthetic heart valve of any example herein, particularly example 44, wherein the outer skirt is urged into contact with surrounding native tissue by the means for displacing when the prosthetic heart valve transitions to the expanded configuration within an anatomy of a patient.
Example 46. The prosthetic heart valve of any example herein, particularly any one of examples 1-45, wherein the valve frame in the compressed and expanded configurations has an annular shape.
Example 47. The prosthetic heart valve of any example herein, particularly any one of examples 1-45, wherein the valve frame in at least the expanded configuration has a tapered or frustoconical shape.
Example 48. The prosthetic heart valve of any example herein, particularly any one of examples 1-47, wherein the outer skirt comprises a foam or fabric material.
Example 49. The prosthetic heart valve of any example herein, particularly any one of examples 1-48, wherein the outer skirt is formed of polyethylene terephthalate (PET), polyurethane (PU), a matrix of PU and polycarbonate (PC), or any combination thereof.
Example 50. The prosthetic heart valve of any example herein, particularly any one of examples 1-49, wherein the outer skirt extends along the axial direction from a first location at or adjacent to the inflow end of the valve frame to a second location at or adjacent to the outflow end of the valve frame.
Example 51. The prosthetic heart valve of any example herein, particularly any one of examples 1-50, further comprising an inner skirt disposed on a radially-inner circumferential surface of the valve frame and coupled thereto.
Example 52. The prosthetic heart valve of any example herein, particularly example 51, wherein a portion of the outer skirt at the inflow end of the valve frame is coupled to the inner skirt.
Example 53. The prosthetic heart valve of any example herein, particularly any one of examples 1-52, wherein the valvular structure is a bicuspid structure with two leaflets.
Example 54. The prosthetic heart valve of any example herein, particularly any one of examples 1-52, wherein the valvular structure is a tricuspid structure with three leaflets.
Example 55. The prosthetic heart valve of any example herein, particularly any one of examples 1-54, wherein the valve frame is formed of a plastically-expandable material or a self-expanding material.
Example 56. The prosthetic heart valve of any example herein, particularly any one of examples 1-55, wherein the prosthetic heart valve is constructed for implantation in an existing heart valve within a patient.
Example 57. The prosthetic heart valve of any example herein, particularly any one of examples 1-56, wherein the prosthetic heart valve is constructed for implantation at an aortic position, a mitral position, a tricuspid position, or a pulmonary position.
Example 58. An assembly comprises a delivery apparatus comprising an elongated shaft; and the prosthetic heart valve of any one of examples 1-57 mounted on the elongated shaft with the valve frame in its compressed configuration for delivery into a patient's body.
Example 59. A method of implanting a prosthetic heart valve in a patient's body comprises inserting an end of a delivery apparatus into vasculature of a patient, the delivery apparatus comprising an elongated shaft, the prosthetic heart valve of any one of examples 1-57 being releasably mounted on the elongated shaft of the delivery apparatus with the valve frame in its compressed configuration; advancing the prosthetic heart valve to an implantation site; and using the delivery apparatus to expand the valve frame of the prosthetic heart valve to its expanded configuration, thereby implanting the prosthetic heart valve at the implantation site.
Example 60. A method of implanting a prosthetic heart valve in a patient's body comprises inserting a distal end of a delivery apparatus into vasculature of a patient, the delivery apparatus comprising an elongated shaft, the prosthetic heart valve of any one of Examples 1-57 being releasably mounted on the elongated shaft of the delivery apparatus with the valve frame in its compressed configuration; advancing the prosthetic heart valve to an implantation site; and deploying the prosthetic heart valve from the delivery apparatus such that the valve frame of the prosthetic heart valve self-expands to its expanded configuration, thereby implanting the prosthetic heart valve at the implantation site.
Example 61. The method of any example herein, particularly any one of examples 59-60, further comprises installing a valve dock at the implantation site, wherein the prosthetic heart valve in the expanded configuration is mounted within the valve dock.
Example 62. The method of any example herein, particularly any one of examples 59-61, wherein the advancing to the implantation site employs transfemoral, transventricular, transapical, or transseptal approaches.
Example 63. The method of any example herein, particularly any one of examples 59-62, wherein transition of the valve frame of the prosthetic heart valve to its expanded configuration causes the sealing frame to transition to its expanded configuration and/or allows the sealing frame to self-expand to its expanded configuration, thereby displacing at least a portion of the outer skirt radially outward and into contact with surrounding native tissue at the implantation site.
Example 64. The method of any example herein, particularly any one of examples 59-62, wherein transition of the valve frame of the prosthetic heart valve to its expanded configuration causes and/or allows the means for displacing to displace at least a portion of the outer skirt radially outward and into contact with surrounding native tissue at the implantation site.
Example 65. The method of any example herein, particularly any one of examples 59-64, wherein the implantation site is within a native heart valve at an aortic position, a mitral position, a tricuspid position, or a pulmonary position.
Example 66. A method of assembling a prosthetic heart valve comprises coupling a sealing frame to a radially-outer surface of a valve frame of the prosthetic heart valve, the valve frame being radially collapsible and expandable between a first compressed configuration and a first expanded configuration, the valve frame having an inflow end and an outflow end separated from the inflow end along an axial direction of the valve frame, the sealing frame being collapsible and expandable between a second compressed configuration corresponding to the first compressed configuration of the valve frame and a second expanded configuration corresponding to the second expanded configuration of the valve frame, the sealing frame having a first axial end, a second axial end, and an intermediate portion between the first and second axial ends along the axial direction, the coupling being such that the first axial end is coupled to the valve frame at the inflow end and the second axial end coupled is to the valve frame at a location between the inflow and outflow ends along the axial direction; and providing an outer skirt surrounding the radially-outer surface of the valve frame, wherein, with the valve frame and the sealing frame in the first and second expanded configurations, respectively, the intermediate portion projects radially outward from the valve frame, thereby displacing at least a portion of the outer skirt radially outward.
Example 67. The method of any example herein, particularly example 66, wherein the coupling the sealing frame to the valve frame is with at least one of the valve frame and the sealing frame in its respective expanded configuration.
Example 68. The method of any example herein, particularly any one of examples 66-67, wherein the coupling the sealing frame to the valve frame is with at least one of the valve frame and the sealing frame in a configuration between its respective expanded and compressed configurations.
Example 69. The method of any example herein, particularly any one of examples 66-68, further comprising, after the coupling the sealing frame to the valve frame and the providing the outer skirt, simultaneously transitioning the valve frame and the sealing frame to the first and second compressed configurations, respectively.
Example 70. The method of any example herein, particularly any one of examples 66-69, further comprising, mounting the prosthetic heart valve in or on an elongated shaft of a delivery apparatus.
Example 71. The method of any example herein, particularly any one of examples 66-70, comprising coupling the outer skirt to the sealing frame.
Example 72. The method of any example herein, particularly example 71, wherein the coupling of the outer skirt to the sealing frame is via one or more sutures.
Example 73. The method of any example herein, particularly any one of examples 71-72, wherein the coupling of the outer skirt is prior to the coupling the sealing frame to the valve frame, such that the coupling the sealing frame to the valve frame and the providing the outer skirt surrounding the radially-outer surface of the valve frame occur at a same time.
Example 74. The method of any example herein, particularly any one of examples 71-72, wherein the coupling of the outer skirt is after the coupling the sealing frame to the valve frame.
Example 75. The method of any example herein, particularly any one of examples 66-70, wherein the providing comprises coupling the outer skirt to the valve frame after the sealing frame has been coupled to the valve frame.
Example 76. The method of any example herein, particularly example 75, wherein the coupling of the outer skirt is via one or more sutures.
Example 77. The method of any example herein, particularly any one of examples 75-76, further comprising, before or after the coupling the outer skirt to the valve frame, coupling the outer skirt to a portion of the sealing frame between the first and second axial ends.
Example 78. The method of any example herein, particularly any one of examples 75-76, wherein after the coupling the outer skirt to the valve frame, the outer skirt is not directly attached to the sealing frame.
Example 79. The method of any example herein, particularly any one of examples 66-78, wherein the sealing frame is formed of a shape memory material.
Example 80. The method of any example herein, particularly example 79, wherein the shape memory material comprises a nickel titanium alloy.
Example 81. The method of any example herein, particularly any one of examples 66-80, wherein the sealing frame in the second expanded configuration has a first height along the axial direction between the first and second axial ends, and the sealing frame in the second compressed configuration has a second height along the axial direction between the first and second axial ends that is greater than the first height.
Example 82. The method of any example herein, particularly example 81, wherein the second height is at least 1.2 times the first height.
Example 83. The method of any example herein, particularly any one of examples 81-82, wherein the second height is about 1.23-1.3 times the first height.
Example 84. The method of any example herein, particularly any one of examples 66-83, wherein, with the valve frame and the sealing frame in the first and second expanded configurations, respectively, the intermediate portion projects outward from the radially-outer surface of the valve frame by an amount along a radial direction of the valve frame that is at least 5% of a diameter of the valve frame.
Example 85. The method of any example herein, particularly example 84, wherein the amount of projection along the radial direction is 6-14% of the diameter of the valve frame.
Example 86. The method of any example herein, particularly any one of examples 66-85, wherein the intermediate portion projects outward from the radially-outer surface of the valve frame by amount 2-4 mm along the radial direction.
Example 87. The method of any example herein, particularly any one of examples 66-86, wherein the sealing frame comprises a continuous unitary structure that spans an entire outer circumference of the radially-outer surface of the valve frame.
Example 88. The method of any example herein, particularly any one of examples 66-87, wherein an inner diameter of the sealing frame at the first axial end is substantially the same as an outer diameter of the valve frame at the inflow end, and an inner diameter of the sealing frame at the second axial end is substantially the same as an outer diameter of the radially-outer surface of the valve frame.
Example 89. The method of any example herein, particularly example 88, wherein the inner diameter of the sealing frame at the first axial end is substantially the same as the inner diameter of the sealing frame at the second axial end.
Example 90. The method of any example herein, particularly any one of examples 66-89, wherein the valve frame comprises a plurality of first struts connected together at respective junctions to form an open-cell lattice structure, each cell being open along the radial direction of the valve frame, junctions at the inflow end forming first apices, and junctions at the outflow end forming second apices.
Example 91. The method of any example herein, particularly example 90, wherein the first apices are offset from the second apices with respect to a circumferential direction of the valve frame.
Example 92. The method of any example herein, particularly any one of examples 90-91, wherein the first axial end of the sealing frame comprises a plurality of first coupling apices, the second axial end of the sealing frame comprises a plurality of second coupling apices, and a plurality of strut portions interconnecting the first and second coupling apices.
Example 93. The method of any example herein, particularly example 92, wherein each of the first and second coupling apices has an eyelet.
Example 94. The method of any example herein, particularly example 93, wherein the coupling the sealing frame to the valve frame comprises coupling each of the first and second coupling apices to a respective portion of the valve frame via one or more sutures through the respective eyelet.
Example 95. The method of any example herein, particularly any one of examples 92-94, wherein at least some of the plurality of strut portions form respective cells that are closed along the axial direction.
Example 96. The method of any example herein, particularly any one of examples 92-95, wherein the intermediate portion of the sealing frame comprises longitudinally-extending coupling portions between adjacent strut portions.
Example 97. The method of any example herein, particularly any one of examples 92-96, wherein each first coupling apex is coupled to a respective one of the first apices of the valve frame, each second coupling apex is coupled to a respective one of the junctions of the first struts of the valve frame, and the first coupling apices are aligned with the second coupling apices, respectively, with respect to a circumferential direction of the valve frame.
Example 98. The method of any example herein, particularly any one of examples 92-97, wherein, after the coupling the sealing frame to the valve frame, the sealing frame extends along the axial direction over at least part of a circumferential row of cells of the lattice structure of the valve frame.
Example 99. The method of any example herein, particularly any one of examples 92-98, wherein, after the coupling the sealing frame to the valve frame, the sealing frame extends along the axial direction over a single circumferential rows of cells of the lattice structure of the valve frame.
Example 100. The method of any example herein, particularly any one of examples 92-99, wherein, after the coupling the sealing frame to the valve frame, the sealing frame extends along the axial direction over at least two circumferential rows of cells of the lattice structure of the valve frame.
Example 101. The method of any example herein, particularly any one of examples 92-100, wherein, after the coupling the sealing frame to the valve frame, the sealing frame extends along the axial direction over three circumferential rows of cells of the lattice structure of the valve frame.
Example 102. The method of any example herein, particularly any one of examples 92-101, wherein at least some of the plurality of strut portions form respective cells that are open along the axial direction.
Example 103. The method of any example herein, particularly example 102, wherein the first coupling apices are offset from the second coupling apices with respect to a circumferential direction of the valve frame.
Example 104. The method of any example herein, particularly any one of examples 90-91, wherein the sealing frame comprises elementary units arrayed around a circumference of the sealing frame, each elementary unit comprising first and second angled strut portions extending from a first apex at the first axial end and third and fourth angled strut portions extending from a second apex at the second axial end, the first apex being aligned with the second apex along the axial direction, the first angled strut portion joining with the third angled strut portion via a first coupling portion, the second angled strut portion joining with the fourth angled strut portion via a second coupling portion, adjacent elementary units in the array being joined together at adjacent coupling portions.
Example 105. The method of any example herein, particularly any one of examples 90-91, wherein the sealing frame comprises elementary units arrayed around a circumference of the sealing frame, each elementary unit comprising first and second angled strut portions extending from a first apex at the first axial end and third and fourth angled strut portions extending from a second apex at the second axial end, the first apex being aligned with the second apex along the axial direction, the first angled strut portion joining with the third angled strut portion via a first longitudinally-extending strut, the second angled strut portion joining with the fourth angled strut portion via a second longitudinally-extending strut, adjacent elementary units in the array being joined together at adjacent longitudinally-extending struts.
Example 106. The method of any example herein, particularly any one of examples 90-91, wherein the sealing frame comprises elementary units arrayed around a circumference of the sealing frame, each elementary unit comprising first and second angled struts extending from a first apex at the first axial end to respective second apices at the second axial end, adjacent elementary units in the array being joined together at adjacent second apices.
Example 107. The method of any example herein, particularly any one of examples 90-91, wherein the sealing frame comprises elementary units arrayed around a circumference of the sealing frame, each elementary unit comprising first and second curved struts extending from a first apex at the first axial end to a coupling portion and third and fourth angled strut portions extending from the coupling portion to respective second apices at the second axial end, adjacent elementary units in the array being joined together at adjacent second apices.
Example 108. The method of any example herein, particularly any one of examples 66-107, wherein, after the providing the outer skirt surrounding the valve frame, the outer skirt extends along the axial direction from at least the inflow end of the valve frame to at least the second axial end of the sealing frame.
Example 109. The method of any example herein, particularly any one of examples 66-108, wherein, after the providing the outer skirt surrounding the valve frame and the coupling the sealing frame to the valve frame, the outer skirt extends along the axial direction from the inflow end of the valve frame farther than the sealing frame.
Example 110. The method of any example herein, particularly any one of examples 66-109, wherein an edge of the outer skirt opposite the inflow end of the valve frame has an undulating pattern with a plurality of axially projecting portions, and the providing the outer skirt surrounding the valve frame is such that each projecting portion is aligned with a corresponding strut junction of the valve frame.
Example 111. The method of any example herein, particularly example 110, wherein, the coupling the sealing frame to the valve frame is such that apices of the sealing frame at the second axial end are aligned with the projecting portions of the undulating pattern, respectively, with respect to a circumferential direction of the valve frame.
Example 112. The method of any example herein, particularly example 110, wherein, the coupling of the sealing frame to the valve frame is such that apices of the sealing frame at the second axial end are offset from the projecting portions of the undulating pattern, respectively, with respect to a circumferential direction of the valve frame.
Example 113. The method of any example herein, particularly any one of examples 66-112, wherein the valve frame in the compressed and expanded configurations has an annular shape.
Example 114. The method of any example herein, particularly any one of examples 66-112, wherein the valve frame in at least the expanded configuration has a tapered or frustoconical shape.
Example 115. The method of any example herein, particularly any one of examples 66-114, wherein the outer skirt comprises a foam or fabric material.
Example 116. The method of any example herein, particularly any one of examples 66-115, wherein the outer skirt is formed of polyethylene terephthalate (PET), polyurethane (PU), a matrix of PU and polycarbonate (PC), or any combination thereof.
Example 117. The method of any example herein, particularly any one of examples 66-116, wherein, after the providing the outer skirt surrounding the valve frame, the outer skirt extends along the axial direction from a first location at or adjacent to the inflow end of the valve frame to a second location at or adjacent to the outflow end of the valve frame.
Example 118. The method of any example herein, particularly any one of examples 66-117, further comprising, prior to or after the coupling the sealing frame to the valve frame, providing an inner skirt over a radially-inner surface of the valve frame.
Example 119. The method of any example herein, particularly example 118, wherein the providing the outer skirt surrounding the valve frame comprises coupling a portion of the outer skirt at the inflow end of the valve frame to a portion of the inner skirt.
Example 120. The method of any example herein, particularly any one of examples 66-119, further comprising, prior to or after the coupling the sealing frame to the valve frame, coupling a valvular structure to the valve frame, the valvular structure comprising a plurality of leaflets.
Example 121. The method of any example herein, particularly example 120, wherein the valvular structure is a bicuspid structure with two leaflets.
Example 122. The method of any example herein, particularly example 120, wherein the valvular structure is a tricuspid structure with three leaflets.
Example 123. The method of any example herein, particularly any one of examples 66-122, wherein the valve frame is formed of a plastically-expandable material or a self-expanding material.

### GENERAL CONSIDERATIONS

All features described herein are independent of one another and, except where structurally impossible, can be used in combination with any other feature described herein. For example, a delivery apparatus 900 as shown in FIG. 9 can be used in combination with any of the prosthetic heart valves described herein.

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present, or problems be solved. The technologies from any example can be combined with the technologies described in any one or more of the other examples.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used herein with reference to the prosthetic heart valve assembly and implantation and structures of the prosthetic heart valve, “proximal” refers to a position, direction, or portion of a component that is closer to the user and a handle of the delivery system or apparatus that is outside the patient, while “distal” refers to a position, direction, or portion of a component that is further away from the user and the handle, and closer to the implantation site. The terms “longitudinal” and “axial” refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic heart valve. Such terms have been used for convenient description, but the disclosed embodiments are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. As used herein, “and/or” means “and” or “or,” as well as “and” and “or.”

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” “right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

In view of the many possible embodiments to which the principles of the disclosed technology may be applied, it should be recognized that the illustrated examples are only preferred examples and should not be taken as limiting the scope of the disclosed technology. Rather, the scope of the invention is defined by the following claims. We therefore claim as our invention all that comes within the scope and spirit of these claims.

1. A prosthetic heart valve comprising:
a valve frame that is radially collapsible and expandable between a first compressed configuration and a first expanded configuration, the valve frame having an inflow end and an outflow end separated from the inflow end along an axial direction of the valve frame;
a valvular structure coupled to the valve frame and comprising a plurality of leaflets within the valve frame;
a sealing frame surrounding a radially-outer surface portion of the valve frame, the sealing frame being collapsible and expandable between a second compressed configuration corresponding to the first compressed configuration of the valve frame and a second expanded configuration corresponding to the second expanded configuration of the valve frame, the sealing frame having a first axial end coupled to the valve frame at the inflow end, a second axial end coupled to the valve frame at a location between the inflow and outflow ends along the axial direction, and an intermediate portion between the first and second axial ends along the axial direction; and
an outer skirt surrounding the sealing frame;
wherein, with the valve frame and the sealing frame in the first and second expanded configurations, respectively, the intermediate portion projects radially outward from the valve frame, thereby displacing at least a portion of the outer skirt radially outward.

2. The prosthetic heart valve of claim 1, wherein, in transitioning between the second compressed configuration and the second expanded configuration within an anatomy of a patient, the intermediate portion of the sealing frame is constructed to urge the outer skirt into contact with surrounding native tissue.

3. The prosthetic heart valve of claim 1, wherein, with the valve frame and the sealing frame in the first and second compressed configurations, respectively, the first axial end, the second axial end, and the intermediate portion of the sealing frame are substantially adjacent to the radially-outer surface portion of the valve frame.

4. The prosthetic heart valve of claim 1, wherein, with the valve frame and the sealing frame in the first and second compressed configurations, respectively, the first axial end, the second axial end, and the intermediate portion of the sealing frame are substantially aligned along a direction substantially parallel to the axial direction.

5. The prosthetic heart valve of claim 1, wherein the sealing frame is formed of a shape memory material.

6. The prosthetic heart valve of claim 5, wherein the shape memory material comprises a nickel titanium alloy.

7. The prosthetic heart valve of claim 1, wherein the sealing frame in the second expanded configuration has a first height along the axial direction between the first and second axial ends, and the sealing frame in the second compressed configuration has a second height along the axial direction between the first and second axial ends that is greater than the first height.

8. The prosthetic heart valve of claim 7, wherein the second height is at least 1.2 times the first height.

9. The prosthetic heart valve of claim 1, wherein, with the valve frame and the sealing frame in the first and second expanded configurations, respectively, the intermediate portion projects outward from the radially-outer surface portion of the valve frame by an amount along a radial direction of the valve frame that is at least 5% of a diameter of the valve frame.

10. The prosthetic heart valve of claim 9, wherein the amount of projection along the radial direction is 6-14% of the diameter of the valve frame.

11. The prosthetic heart valve of claim 1, wherein the intermediate portion projects outward from the radially-outer surface portion of the valve frame by amount 2-4 mm along the radial direction.

12. The prosthetic heart valve of claim 1, wherein the sealing frame comprises a continuous unitary structure that spans an entire outer circumference of the radially-outer surface portion of the valve frame.

13. The prosthetic heart valve of claim 1, wherein the valve frame comprises a plurality of first struts connected together at respective junctions to form an open-cell lattice structure, each cell being open along the radial direction of the valve frame, junctions at the inflow end forming first apices, and junctions at the outflow end forming second apices.

14. The prosthetic heart valve of claim 13, wherein the first axial end of the sealing frame comprises a plurality of first coupling apices, the second axial end of the sealing frame comprises a plurality of second coupling apices, and a plurality of strut portions interconnecting the first and second coupling apices.

15. The prosthetic heart valve of claim 14, wherein the intermediate portion of the sealing frame comprises longitudinally-extending coupling portions between adjacent strut portions.

16. The prosthetic heart valve of claim 13, wherein the sealing frame extends along the axial direction over at least part of a circumferential row of cells of the lattice structure of the valve frame.

17. The prosthetic heart valve of claim 13, wherein the sealing frame extends along the axial direction over one or more circumferential rows of cells of the lattice structure of the valve frame.

18. An assembly comprising:
a delivery apparatus comprising an elongated shaft; and
the prosthetic heart valve mounted on the elongated shaft, the prosthetic heart valve comprising a valve frame in a compressed configuration for delivery into a patient's body, the valve frame being radially collapsible and expandable between the first compressed configuration and a first expanded configuration, the valve frame having an inflow end and an outflow end separated from the inflow end along an axial direction of the valve frame, the valve frame,
the prosthetic heart valve further comprising
a valvular structure coupled to the valve frame and comprising a plurality of leaflets within the valve frame;
a sealing frame surrounding a radially-outer surface portion of the valve frame, the sealing frame being collapsible and expandable between a second compressed configuration corresponding to the first compressed configuration of the valve frame and a second expanded configuration corresponding to the second expanded configuration of the valve frame, the sealing frame having a first axial end coupled to the valve frame at the inflow end, a second axial end coupled to the valve frame at a location between the inflow and outflow ends along the axial direction, and an intermediate portion between the first and second axial ends along the axial direction, and
an outer skirt surrounding the sealing frame;
wherein, with the valve frame and the sealing frame in the first and second expanded configurations, respectively, the intermediate portion projects radially outward from the valve frame, thereby displacing at least a portion of the outer skirt radially outward.

19. A method of implanting a prosthetic heart valve in a patient's body, the method comprising:
inserting an end of a delivery apparatus into vasculature of a patient, the delivery apparatus comprising an elongated shaft and the prosthetic heart valve of claim 1 releasably mounted on the elongated shaft with the valve frame in the compressed configuration;
advancing the prosthetic heart valve to an implantation site; and
deploying the prosthetic heart valve at the implantation site.

20. The method of claim 19, wherein deploying the prosthetic heart valve comprises expanding the valve frame using the delivery apparatus.
